(19) United States
(12) Patent Application Publication
(10) Pub. No.: US 2011/0319592 A1
(43) Pub. Date: Dec. 29, 2011

(54) Process for the Purification of Antibodies Using Affinity Resins Comprising Specific Ligands

(75) Inventors: Phaedria Marie St. Hilaire, Kobenhavn NV (DK); Anders Laerke Nielsen, Kobenhavn S (DK)

(73) Assignee: Novo Nordisk A/S, Bagsvaerd (DK)

(21) Appl. No.: 13/129,009

(22) PCT Filed: Nov. 13, 2009
(86) PCT No.: PCT/EP09/65144

§ 371 Date: Aug. 30, 2011

Related U.S. Application Data
(60) Provisional application No. 61/116,064, filed on Nov. 19, 2008.

(30) Foreign Application Priority Data
Nov. 13, 2008 (EP) 08169013.3

(51) Int. Cl. C07K 9/00; C07K 1/22; C07D 333/70; C07K 5/097; C07D 409/12; C07K 16/44; C08B 37/12

(52) U.S. Cl. 530/322; 530/389.8; 536/53; 530/331; 549/57

(57) ABSTRACT

The present invention relates to a novel process for the purification of antibodies, e.g. monoclonal antibodies. The process utilizes an affinity resin comprising a solid phase material having immobilized thereto one or more low-molecular weight synthetic ligands. The affinity resins enable the separation of antibodies from even closely related proteins.

### FIELD OF THE INVENTION

The present invention relates to a novel process for the purification of antibodies, e.g. monoclonal antibodies. The process utilizes an affinity resin comprising a solid phase material having immobilized thereto one or more low-molecular weight synthetic ligands. The affinity resins enable the separation of antibodies from even closely related proteins.

### BACKGROUND OF THE INVENTION

In recent years, monoclonal antibodies (mAbs) have become the primary focus for many major biotech companies. mAbs are being tested as biopharmaceuticals for treating a wide area of diseases, with special emphasis on immune-, and auto-immune diseases and cancer. As the number of mAb drugs on the market is expected to increase dramatically in the future, the need for reducing the production costs is increasing accordingly. In current day production the purification step in the mAb production is responsible for approx. 70% of the production costs.

Currently antibodies can be purified from fermentation supernatants by various processes, e.g. conventional chromatography, affinity chromatography with either protein ligands or small molecule ligands.

Conventional chromatography involving multiple steps suffers from one or more of the following: low overall yield, high buffer consumption, long process time and large investment in process equipment, increased labour.

Affinity resins with protein ligands result in higher yield, lower buffer consumption, and reduced investment in process equipment. However, affinity resins with protein ligands are very costly to manufacture as well as less chemically and conformationally stable compared to small synthetic ligands and inherently hold a risk that the purified antibodies be contaminated with protein fragments from the protein ligands or other biological matter from the production of the affinity resin with protein ligands.

U.S. Pat. No. 6,117,996 discloses affinity ligand-matrix conjugates comprising a ligand with the general formula,

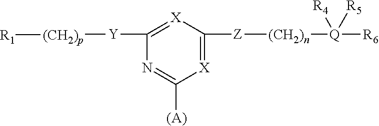

The ligand is attached to a support matrix in position (A), optionally through a spacer arm interposed between the matrix and ligand. The purification of proteinaceous materials such as e.g. insulins, Factor VII, human Growth Hormone or analogues, antibodies such as immunoglobulins, derivatives and fragments thereof and precursors using such affinity ligand-matrix conjugates is also disclosed.

U.S. Pat. No. 6,498,236 discloses affinity ligands with the general formula,

(H₂N—X₁-Thr-X₂—CO)n-R

for the purification of immunoglobulins.

WO 2006/066598 discloses affinity ligands comprising one or more hydrophobic functional group(s) and one or more cationic functional groups(s) for purification of monoclonal antibodies.

U.S. Pat. No. 6,207,807 discloses tripeptide affinity ligands comprising for the purification of immunoglobulin. The tetrameric tripeptide (Arg-Thr-Tyr)₄ was identified from the screening of a combinatorial library (Ref: Fassina et al. JOURNAL OF MOLECULAR RECOGNITION, VOL. 9,564-569 (1996)) and the affinity resins derived used to isolate immunoglobulins from Serum. For improved stability, an all D-aminoacid version of the muntimeric tripeptide was prepared and used for purification of monoclonal antibodies (ref: Fassina et al, Journal of Immunological Methods 333 (2008) 126-138).

D'Agostino et al [(2008); Affinity purification of IgG monoclonal antibodies using the D-PAM synthetic ligand: Chromatographic comparison with protein A and thermodynamic investigation of the D-PAM/IgG interaction. Journal of Immunological Methods, Vol. 333, Issue 1-2, Pages 126-138] only describes a multimeric molecule for the purification of antibodies. Wherein binding species (such as linear tripeptide, Arg-Thr-Tyr) is coupled to a trilysine scaffold in order to create a multivalent ligand with increased avidity for the antibodies. However the trilysine core scaffold does not interact with the antibody.

Indeed, the prior art covering small molecule affinity resins do overcome some of the drawbacks of protein ligand affinity resins. However, there is still a need for novel procedures and materials for the purification of antibodies, in particular a need for cheaper, more base stable, and more selective affinity resins than those disclosed in prior art. In additions resins that allow mild elution conditions that do not affect the activity of the target protein are desired.

### SUMMARY OF THE INVENTION

The present invention relates to providing affinity resins comprising novel synthetic affinity ligands that are selective for antibodies, in particular monoclonal antibodies, and are not based on protein ligands and which are cheap and base stable.

### BRIEF DESCRIPTION OF THE DRAWINGS

FIG. 1. A schematic outline of the general structure of the trimeric ligands (A) and the tetrameric ligands (B).

FIG. 2. Principal component plot of the 229,957 virtual ligand structures in the first two principal components. The data points are coloured according to the scaffold and the black squares are 28 previously identified binders used for the library design (WO 2006/066598).

FIG. 3. All library members plotted in the same two principal components used in FIG. 2. It is seen that the 770 selected ligand structures span a significant part of the chemical space spanned by the full virtual library.

FIG. 4. Average fluorescence values for the 679 identified ligands. It is seen that certain ligands exhibit fluorescence values significantly above the background noise level.

FIG. 5. A plot of all ligands with an averaged fluorescence value above 40. The points are coloured according to fluorescence level: green: 40-70, yellow: 70-100, red: 100-200, and blue: >200. The high-affinity ligands are concentrated in the area of the chemical space indicated by the smaller ellipsis.

FIG. 6. The frequency of the 5 scaffolds (A), the frequency of the 14 building block 1's (B), the frequency of the 11 building block 2's (C) in the hits.

FIG. 7. Chromatogram of affinity purification of human IgG4.

FIG. 8. Gel analysis: Molecular weight standard (Lane 1 from right), IgG4 reference (2), harvest (3), flow-through (4), wash (5), elution (6).

FIG. 9. Analytical HPLC chromatogram of the eluate from the column.

### DETAILED DESCRIPTION OF THE INVENTION

The present invention relates to a process for purification of an antibody involving the use of affinity ligands and affinity resins wherein the ligands are specific binding partners of an antibody and can therefore be used for the purification of an antibody.

More specifically, the present invention relates to providing affinity resins comprising novel synthetic affinity ligands that are selective for antibodies, in particular monoclonal antibodies, and are not based on protein ligands and which are cheap and base stable.

The affinity resin is a solid phase material (see further below) having covalently immobilized thereto ligands that have a high specificity towards the antibody in question.

Accordingly, one embodiment of present invention provides a process for purification of an antibody said process comprising the steps of:

(a) contacting the solution or suspension containing the antibody with an affinity resin under conditions which facilitate binding of a portion of said antibody to said affinity resin;

(b) optionally washing said affinity resin containing bound antibody with a washing buffer; and

(c) eluting said affinity resin containing bound antibody with an elution buffer, and collecting a purified antibody as an eluate.

In one embodiment of present invention, the affinity resin is a solid phase material having covalently immobilized thereto one or more ligands of the general formula (I),

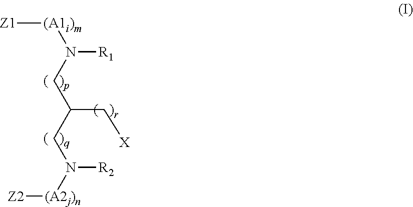

wherein

i=1,2, . . . , m, and j=1,2, . . . , n;

n and m are independently an integer in the range of 0-3, with the proviso that the sum n+m is in the range of 1-4;

p, q, and r are independently an integer in the range of 0-6;

A11, . . . , A1m, and A21, . . . , A2n are independently selected from α-amino acid moieties, β-amino acid moieties, α-amino sulphonic acid moieties, and β-amino sulphonic acid moieties;

Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl;

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl and Hal is a halogen; and

the total molecular weight of said ligand (excluding “X” and any linker) being 200-2000 g/mol.

It has surprisingly been found that the scaffolds based on e.g. α,ω-diamino-carboxylic acids and similar types, such as α,β-diamino-propionic acid (p=1, q=r=0), α,γ-diamino-butyric acid, and α,δ-diamino-pentoic acid, in particular α,β-diamino-propionic acid (p=1, q=r=0), offer interesting classes of ligands which have excellent binding properties towards antibodies, and which further represents a specific binding to antibodies compared to other proteins present in, e.g., cell culture supernatants or plasma.

Additionally, the ligand excluding “X” and any linker preferably has a molecular weight of more than 200 Da, such as more than 300 Da, for example more than 400 Da, such as more than 500 Da, for example more than 600 Da, such as more than 700 Da, for example a molecular weight of more than 800 Da. Independently thereof, the ligand preferably has a molecular weight of less than 5000 Da, such as less than 4000 Da, for example less than 3000 Da, such as less than 2500 Da, for example less than 2000 Da, such as less than 1500 Da, for example a molecular weight of less than 1000 Da.

In one embodiment of present invention, each of Z1-(A1i)ₘ-N(R1)- and Z2-(A2j)ₙ-N(R2)- represents an organic moiety of a molecular weight of 50-500 g/mol, wherein the total molecular weight of the ligand is 250-1500 g/mol, such as 300-1200 g/mol, e.g. 350-1000 g/mol.

In one embodiment of present invention, A11, . . . , A1m, and A21, . . . , A2n are independently selected from α-amino acid moieties and β-amino acid moieties, in particular from α-amino acid moieties.

In one embodiment of present invention, A11, . . . , A1m and A21, . . . , A2n are independently selected from the following amino acids in either of the L and D forms: glycine, proline, arginine, tyrosine, glutamine, valine, cysteine, histidine, and leucine, in particular from glycine, L-proline, L-arginine, L-tyrosine, D-glutamine, D-tyrosine, L-valine, L-cysteine, L-histidine, and L-leucine.

In one embodiment of present invention, at least one of A11, . . . , A1m and A21, . . . , A2n is selected from glycine, L-proline, L-arginine, L-tyrosine, D-glutamine, D-tyrosine, L-valine, L-cysteine, L-histidine, and L-leucine.

In one embodiment of present invention, A1₁, . . . , A1ₘ, A2₁, . . . , A2ₙ include at least one amino acid moiety selected from arginine, tyrosine and glutamine, in particular at least one amino acid moiety selected from L-arginine, L-tyrosine and D-glutamine.

In one embodiment of present invention, Z1 and Z2 are independently selected from hydrogen, C₁₋₆ alkyl, carboxylic acid moieties, and sulphonic acid moieties.

In one embodiment of present invention, the variant hereof, Z1 and Z2 include at least one carboxylic acid moiety, or sulphonic acid moiety, in particular at least one carboxylic acid moiety.

In one embodiment of present invention, Z1 and Z2 are independently selected from hydrogen, C₁₋₆ alkyl, 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl.

In one embodiment of present invention Z1 and Z2 are independently selected from 3,5-di-tert-butyl-4-hydroxy-benzoyl and thianaphthenyl.

In one embodiment of present invention includes those ligands wherein at least one of Z1 and Z2 is thianaphthene-2-carbonyl.

In one embodiment of present invention with respect to the number of amino acids/amino sulphonic acids, n is preferably 0-2, such as 0-1, in particular 0, and m is preferably 1-3, such as 2-3, in particular 2. The sum n+m is preferably 2-4, such as 2-3, in particular 2 or 3.

In one embodiment of present invention, when n is 0, Z2 is preferably selected from carboxylic acid moieties and sulphonic acid moieties. Also, when m is 0, Z1 is preferably selected from carboxylic acid moieties and sulphonic acid moieties.

In one embodiment of present invention with respect to the number of carbon atoms in the scaffold, p is preferably 0-3, such as 0-2, such as 1-2, in particular 2, and q is preferably 0-3, such as 0-2, in particular 0 or 1. The sum p+q is preferably 1-7, such as 2-5, in particular 2 or 3. Independently thereof r is preferably 0-6, such as 0-4, such as 0-2, in particular 0 or 1, more particular 0.

In one embodiment of present invention, further variants with respect to the number of carbon atoms in the scaffold, (p,q) is (0,1), (0,2), (0,3), (0,4), (1,0), (2,0), (3,0), or (4,0).

In one embodiment of present invention, further variants hereof, r is preferably 0.

As mentioned above, X is the reactive group used for attaching the ligand to the solid phase material, either directly or via a linker (see further below). In one embodiment of present invention the linker is attached to the scaffold via a segment —CON(R)— (in particular —NHCO—), where the carbonyl is a part of the scaffold representing “X”, because this will render it possible to prepare the ligand by standard methodologies known from solid phase peptide synthesis.

In one embodiment of present invention, X is typically selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃)0, wherein R is selected from optionally substituted C₁₋₁₂-alkyl, and Hal is a halogen.

A particularly interesting meaning for X is COOH.

In one embodiment of present invention the affinity resins comprising a solid phase material having a ligand of the general formula (I) attached optionally via a linker, wherein

A11, . . . , A1m and A21, . . . , A2n are independently selected from glycine, proline, arginine, tyrosine, glutamine, tyrosine, valine, cysteine, histidine, and leucine, in particular from glycine, L-proline, L-arginine, L-tyrosine, D-glutamine, D-tyrosine, L-valine, L-cysteine, L-histidine, and L-leucine;

Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl;

R1 and R2 are independently selected from hydrogen and C₁₋₆-alkyl; and

m is 0 or 1, n is 0 or 1, (p,q) is (0,1), (0,2), (0,3), (0,4), (1,0), (2,0), (3,0), or (4,0), and r is 0;

In one embodiment of present invention, the ligand has the general formula (II),

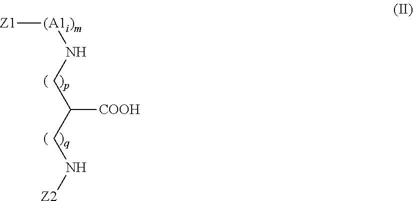

wherein Z1, Z2, A11, . . . , A1m, p and q are as defined above for general formula (I), including the embodiments described for this general formula.

In one embodiment of present invention particularly preferred are those ligands of general formula (II), wherein

p and q are independently an integer in the range of 0-6;

i=1,2, . . . , m, and m is an integer in the range of 1-4, such as 1-3, e.g. 1-2;

A11, . . . , A1m are independently selected from glycine, proline, arginine, tyrosine, glutamine, tyrosine, valine, cysteine, histidine, and leucine, in particular glycine, L-proline, L-arginine, L-tyrosine, D-glutamine, D-tyrosine, L-valine, L-cysteine, L-histidine, and L-leucine; and

Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl.

In one embodiment of present invention particularly preferred are those ligands of general formula (II), wherein

p and q are independently an integer in the range of 0-6;

i=1,2, . . . , m, and m is an integer in the range of 1-4, such as 1-3, e.g. 1-2;

A11, . . . , A1m are independently selected from L-proline, L-arginine, L-tyrosine, D-glutamine, D-tyrosine, L-valine; and

Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl.

In one embodiment of present invention, the ligand has the general formula (I), where A2₁ is preferably selected from arginine, phenylalanine, tyrosine, isoleucine, and lysine, and A2₂ is selected from arginine, phenylalanine, isoleucine, proline, tyrosine, and tryptophan.

In one embodiment of present invention, the ligand has a structure selected from the following ligands, Nos. (1)-(12):

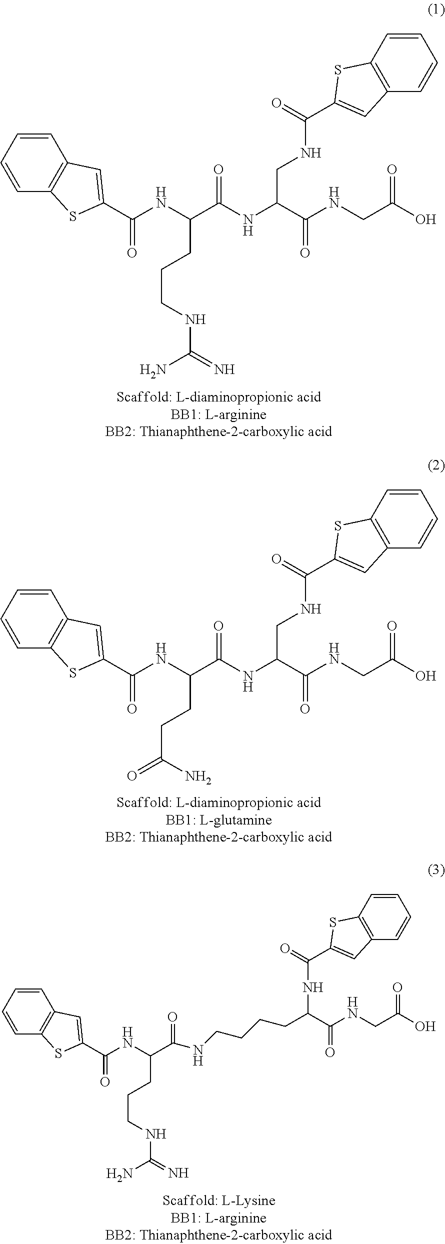

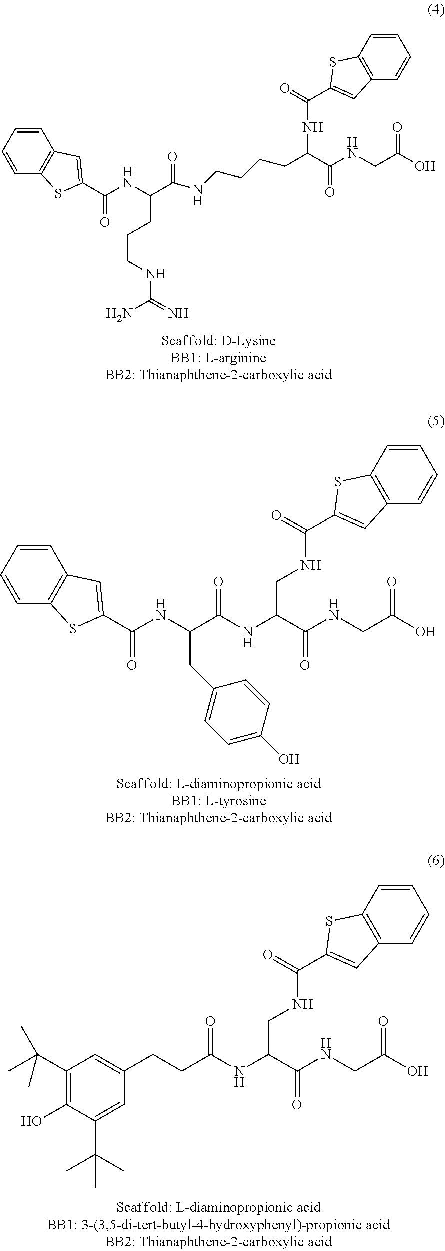

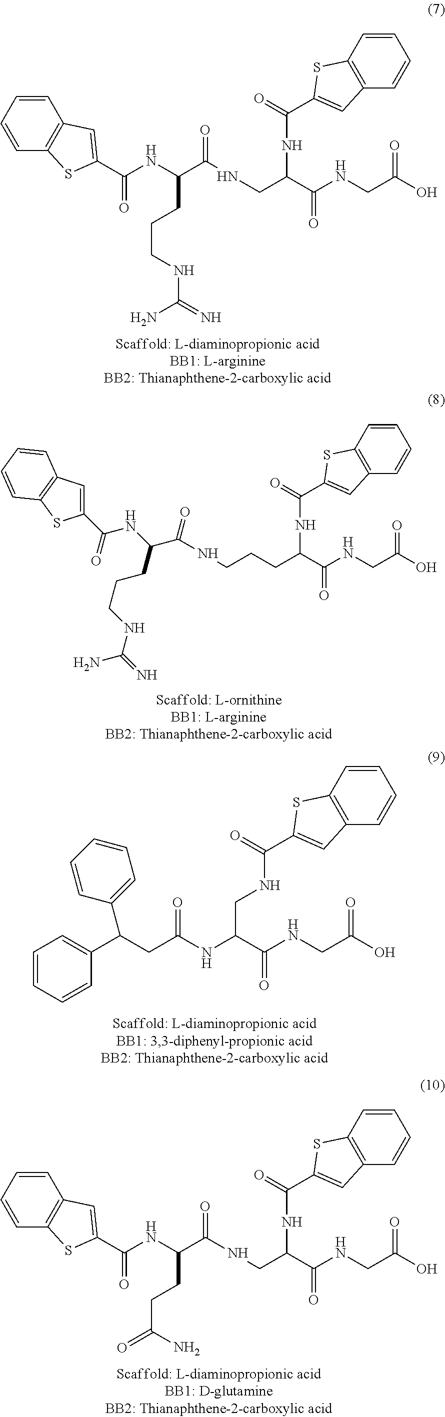

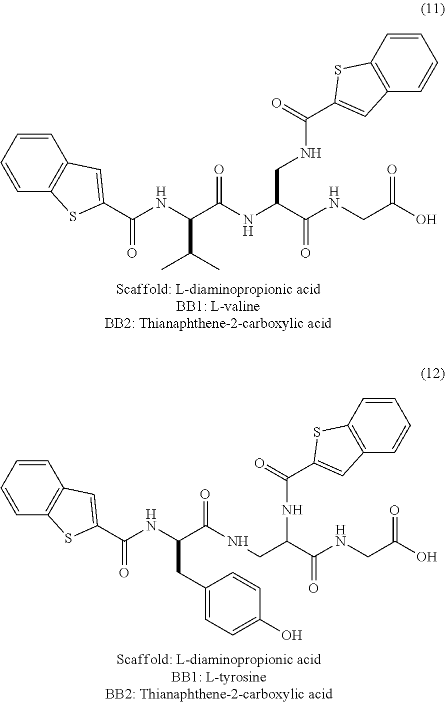

In one embodiment of present invention, the ligand has the general formula (III),

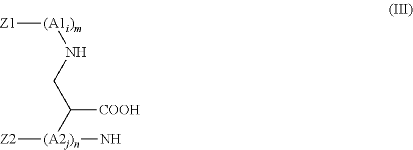

wherein Z1, Z2, A11, . . . , A1m and A21, . . . , A2n, are as defined above for general formulae (I) and (II), including the embodiments described for these general formulae.

In one embodiment of present invention the ligand of general formula (III), Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl, acetyl, 2-phenyl-2-cyclopentyl acetyl, 2-quinaldoyl, indole-2-carbonyl, phthalamoyl, 4-hydroxy-3-(morpholinomethyl)benzoyl, 2-phenyl-4-quinoline carbonyl, 4,4-Bis(4-hydroxyphenyl)-N-valeroyl, 4-sulfamoylbenzoyl, hydroxyquinaldoyl, and 5-acetamido-2-hydroxy benzoyl.

In one embodiment of present invention the ligand of general formula (III), Z1 and Z2 are independently selected from 3,5-dimethoxy-2-naphthyl, thianaphthenyl, 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, naphthyl, 2-naphthyl acetyl, phenyl-acetyl, acetyl, 2-phenyl-2-cyclopentyl acetyl, 2-quinaldoyl, indole-2-carbonyl, phthalamoyl, 4-hydroxy-3-(morpholinomethyl)benzoyl, 2-phenyl-4-quinoline carbonyl, 4,4-Bis(4-hydroxyphenyl)-N-valeroyl, 4-sulfamoylbenzoyl, hydroxyquinaldoyl, and 5-acetamido-2-hydroxy benzoyl.

The ligands described herein include enriched or resolved optical isomers at any or all asymmetric atoms as are apparent from the description or depiction herein. Both racemic and diastereomeric mixtures, as well as the individual optical isomers can be isolated or synthesized so as to be substantially free of their enantiomeric or diastereomeric counterparts, and these are all within the scope of the invention.

Definitions

“α-Amino acids moieties” refer to naturally occurring and synthetic amino acids (including the essential amino acids) wherein the amino group is covalently bonded to the α-carbon. When present in the ligands of the invention, the α-amino acid moieties are present as a —N(R)—X—C(═O)— fragment where X represent the α-carbon and any side chain(s).

“β-Amino acids moieties” refer to naturally occurring and synthetic amino acids wherein the amino group is covalently bonded to the β-carbon. When present in the ligands of the invention, the β-amino acid moieties are present as a —N(R)—X—C(═O)— fragment where X represent the α- and β-carbons and any side chain(s).

“α-amino sulphonic acid moieties” corresponds to “α-amino acids moieties” wherein the carbonyl group (—C(═O)—) has been replaced with a sulphonic group (—S(═O)₂—). When present in the ligands of the invention, the α-amino sulphonic acid moieties are present as a —N(R)—X—S(═O)₂— fragment where X represent the α-carbon and any side chain(s).

“β-amino sulphonic acid moieties” corresponds to “β-amino acids moieties” wherein the carbonyl group (—C(═O)—) has been replaced with a sulphonic group (—S(═O)₂—). When present in the ligands of the invention, the β-amino sulphonic acid moieties are present as a —N(R)—X—S(═O)₂— fragment where X represent the α- and β-carbons and any side chain(s).

The term “essential amino acid” refers to any one of the 20 genetically encoded L-α-amino acids and their stereoisomeric D-α-amino acids. Hence, the term “amino acid moieties” within the scope of the present invention is used in its broadest sense and is meant to include naturally-occurring L-amino acids thereof. The commonly used one- and three-letter abbreviations for naturally-occurring amino acids are used herein (Lehninger, Biochemistry, 2d ed., pp. 71-92, (Worth Publishers: New York, 1975). The term also includes D-amino acids (and residues thereof) as well as chemically-modified amino acids, such as amino acid analogues, including naturally-occurring amino acids that are not usually incorporated into proteins, such as norleucine, as well as chemically-synthesized compounds having properties known in the art to be characteristic of an amino acid.

Examples of amino acids that are generally capable of being incorporated into ligands according to the present invention as “amino acid moieties” are listed herein below:

Glycyl (GLY); aminopolycarboxylic acids, e.g., aspartic acid (ASP), p-hydroxyaspartic acid, glutamic acid (GLU), β-hydroxyglutamic acid, β-methylaspartic acid, β-methylglutamic acid, β,β-dimethylaspartic acid, γ-hydroxyglutamic acid, β,γ-dihydroxyglutamic acid, β-phenylglutamic acid, γ-methyleneglutamic acid, 3-aminoadipic acid, 2-aminopimelic acid, 2-aminosuberic acid and 2-aminosebacic acid residues; glutamine (GLN); asparagine (ASN); arginine (ARG), lysine (LYS), β-aminoalanine, γ-aminobutyrine, ornithine (ORN), citruline, homoarginine, homocitrulline, 5-hydroxy-2,6-diaminohexanoic acid, diaminobutyric acid; histidine (HIS); α,α′-diaminosuccinic acid, α,α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid residues; proline (PRO), 4- or 3-hydroxy-2-pyrrolidine-carboxylic acid, γ-methylproline, pipecolic acid, 5-hydroxypipecolic acid, —N[CH₂]₂CO—, azetidine-2-carboxylic acid; alanine (ALA), valine (VAL), leucine (LEU), allylglycine, butyrine, norvaline, norleucine (NLE), heptyline, α-methylserine, α-amino-α-methyl-γ-hydroxyvaleric acid, α-amino-α-methyl-6-hydroxyvaleric acid, α-amino-α-methyl-ε-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, α-aminodiisoamyacetic acid, α-methylaspartic acid, α-methylglutamic acid, 1-aminocyclopropane-1-carboxylic acid; isoleucine (ILE), alloisoleucine, tert-leucine, β-methyltryptophan; α-amino-α-ethyl-β-phenylpropionic acid; β-phenylserinyl; serine (SER), β-hydroxyleucine, β-hydroxynorleucine, β-hydroxynorvaline, α-amino-α-hydroxystearic acid; homoserine, γ-hydroxynorvaline, δ-hydroxynorvaline, ε-hydroxynorleucine; canavinyl, canalinyl; γ-hydroxyornithinyl; 2-hexosaminic acid, D-glucosaminic acid, D-galactosaminic acid; α-amino-β-thiols, penicillamine, β-thiolnorvaline, β-thiolbutyrine; cysteine (CYS); homocystine; β-phenylmethionine; methionine (MET); S-allyl-(L)-cysteine sulfoxide; 2-thiolhistidine; cystathionine; phenylalanine (PHE), tryptophan (TRP), α-aminophenylacetic acid, α-aminocyclohexylacetic acid, α-amino-β-cyclohexylpropionic acid; aryl-, C₁₋₆-alkyl-, hydroxyl-, halogen-, guanidine-, oxyalkylether-, nitro-, sulphur- or halo-substituted phenyl (e.g., tyrosine (TYR), methyltyrosine and o-chloro-, p-chloro-, 3,4-dicloro, o-, m- or p-methyl-, 2,4,6-trimethyl-, 2-ethoxy-5-nitro, 2-hydroxy-5-nitro and p-nitro-phenylalanine); furyl-, thienyl-, pyridyl-, pyrimidinyl-, purine or naphthylalanines; kynurenine, 3-hydroxykynurenine, 2-hydroxytryptophan, 4-carboxytryptophan; sarcosine (N-methylglycine; SAR), N-benzylglycine, N-methylalanine, N-benzylalanine, N-methylphenylalanine, N-benzylphenylalanine, N-methylvaline and N-benzylvaline; threonine (THR), allothreonine, phosphoserine, phosphothreonine.

In the present context, the terms “C₁₋₁₂-alkyl” and “C₁₋₆-alkyl” are intended to mean a linear, cyclic or branched hydrocarbon group having 1 to 12 carbon atoms and 1 to 6 carbon atoms, respectively, such as methyl, ethyl, propyl, iso-propyl, pentyl, cyclopentyl, hexyl, cyclohexyl. The term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Although the term “C₃₋₁₂-cycloalkyl” is encompassed by the term “C₁₋₁₂-alkyl”, it refers specifically to the mono- and bicyclic counterparts, including alkyl groups having exo-cyclic atoms, e.g. cyclohexyl-methyl.

Similarly, the terms “C₂₋₁₂-alkenyl” and “C₂₋₆-alkenyl” are intended to cover linear, cyclic or branched hydrocarbon groups having 2 to 12 carbon atoms and 2 to 6 carbon atoms, respectively, and comprising (at least) one unsaturated bond. Examples of alkenyl groups are vinyl, allyl, butenyl, pentenyl, hexenyl, heptenyl, octenyl, heptadecaenyl. Preferred examples of alkenyl are vinyl, allyl, butenyl, especially allyl.

Although the term “C₃₋₁₂-cycloalkenyl” is encompassed by the term “C₂₋₁₂-alkenyl”, it refers specifically to the mono- and bicyclic counterparts, including alkenyl groups having exo-cyclic atoms, e.g. cyclohexenyl-methyl.

Similarly, the terms “C₂₋₁₂-alkynyl” and “C₂₋₆-alkynyl” are intended to cover linear, cyclic or branched hydrocarbon groups having 2 to 12 carbon atoms and 2 to 6 carbon atoms, respectively, and comprising (at least) one triple bond.

The term “C₁₋₆-alkoxy” is intended to mean “C₁₋₆-alkyl-O”.

In the present context, i.e. in connection with the terms “alkyl”, “alkoxy”, “alkenyl”, “alkynyl”, and the like, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, —N(C₁₋₄-alkyl)₃⁺, carbamoyl, mono- and di(C₁₋₆-alkyl)aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)amino-carbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, biphenyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydropyrimidine, diazepane, diazocane, pyrrolidine, piperidine, azepane, azocane, aziridine, azirine, azetidine, pyroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), oxide (only relevant as the N-oxide), carboxy, C₁₋₆-alkoxycarbonyl, C₁₋₆-alkylcarbonyl, formyl, aryl, aryloxy, arylamino, aryloxycarbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Carboxylic acid moieties refer to moieties which are included as Z1 and Z2 as Q-C(═O)—, where Q is selected from optionally substituted C₁₋₁₂-alkyl, optionally substituted C₂₋₁₂-alkenyl, optionally substituted C₂₋₁₂-alkynyl, optionally substituted aryl, optionally substituted heteroaryl, and optionally substituted heterocyclyl.

Sulphonic acid moieties refer to moieties which are included as Z1 and Z2 as Q-S(═O)₂—, where Q is selected from optionally substituted C₁₋₁₂-alkyl, optionally substituted C₂₋₁₂-alkenyl, optionally substituted C₂₋₁₂-alkynyl, optionally substituted aryl, optionally substituted heteroaryl, and optionally substituted heterocyclyl.

When used herein, the expression “organic moiety” (and “organic moieties”) is intended to mean a molecular fragment comprising one or more carbon atoms and one or more hydrogen (H), oxygen (O), nitrogen (N), sulphur (S), bromine (Br), chlorine (Cl), fluorine (F), or phosphor (P) atoms covalently bonded.

In certain aspects of the invention, each of the organic moieties Z1-(A1)ₙ-X1 and Z2-(A2)ₘ-X2 typically have the general formula CₓH_{y}O_{z}NₖSₗBrₘClₙFₚP_{q} wherein 0≦x≦15, 0≦y≦2x+1, 0≦z≦x, 0≦k≦x, 0≦l≦x, 0≦m≦3x, 0≦n≦3x, 0≦p≦3x, 0≦q≦x and 50≦12x+y+16z+14k+32l+80m+35n+19p+31q≦500.

Solid Phase Material

As mentioned above, the affinity resin is a solid phase material substituted having immobilized thereto one or more synthetic ligands. The solid phase material (sometimes also referred to as “a matrix” or “a polymer matrix”) may in principle be selected from a broad range of the materials conventionally use for chromatographic purposes and for peptides synthesis. Examples of such materials are described below.

The ligand is covalently immobilized to a solid phase material such as a porous, inorganic matrix or a polymer matrix, optionally in cross-linked and/or beaded form or in a monolithic porous entity. Preferably, the pores of the polymer matrix are sufficiently wide for the target protein to diffuse through said pores and interact with the ligand on the inner surface of the pores. For an antibody with molar mass approx. 150 kDa an average pore diameter of 50-150 nm is preferred, such as approx. 90 nm.

The beaded and optionally cross-linked polymer matrix in one embodiment comprises a plurality of hydrophilic moieties. The hydrophilic moieties can be polymer chains which, when cross-linked, form the cross-linked polymer matrix. Examples include e.g. polyethylene glycol moieties, polyamine moieties, polyvinylamine moieties, and polyol moieties.

In one embodiment of present invention, the core and/or the surface of a beaded polymer matrix comprises a polymeric material selected from the group consisting of polyvinyls, polyacrylates, polyacrylamides, polystyrenes, polyesters and polyamides.

The beaded polymer matrix can also be selected from the group consisting of PS, POEPS, POEPOP, SPOCC, PEGA, CLEAR, Expansin, Polyamide, Jandagel, PS-BDODMA, PS-HDODA, PS-TTEGDA, PS-TEGDA, GDMA-PMMA, PS-TRPGDA, ArgoGel, Argopore resins, ULTRAMINE, crosslinked LUPAMINE, high capacity PEGA, Silica, Fractogel, Sephadex, Sepharose, Glass beads, crosslinked polyacrylates, and derivatives of the aforementioned; in particular, the polymer matrix is selected from the group consisting of SPOCC, PEGA, HYDRA, POEPOP, PEG-polyacrylate copolymers, polyether-polyamine copolymers, and cross-linked polyethylene di-amines.

Apart from the above-mentioned examples, any material capable of forming a polymer matrix can in principle be used in the production of beads of the invention. Preferably, the core material of a bead is polymeric. In some embodiments, the core comprises or consists of hydrophilic polymeric material. In other embodiments, the core comprises or consists of hydrophobic polymeric material. In some embodiments, the surface of the beads comprises or consists of the same material as the core.

Resins useful for large-scale applications may be one of the above mentioned or other commercial resins such as Sephadex™, Sepharose™, Fractogel™, CIMGEL™, Toyopearl, HEMA™, crosslinked agarose, and macroporous polystyrene or polyacrylate. The matrix may also be of a mainly inorganic nature, such as macroporous glass or clay minerals, or combinations of resins and inorganics, such as Ceramic HyperD™ or silica gel.

Polymer beads according to the invention can be prepared from a variety of polymerisable monomers, including styrenes, acrylates and unsaturated chlorides, esters, acetates, amides and alcohols, including, but not limited to, polystyrene (including high density polystyrene latexes such as brominated polystyrene), polymethylmethacrylate and other polyacrylic acids, polyacrylonitrile, polyacrylamide, polyacrolein, polydimethylsiloxane, polybutadiene, polyisoprene, polyurethane, polyvinylacetate, polyvinylchloride, polyvinylpyridine, polyvinylbenzylchloride, polyvinyltoluene, polyvinylidenechloride and polydivinylbenzene. In other embodiments, the beads are prepared from styrene monomers or PEG based macro-monomers. The polymer is in preferred embodiments selected from the group consisting of polyethers, polyvinyls, polyacrylates, polymethacrylates, polyacylamides, polyurethanes, polyacrylamides, polystyrenes, polycarbonates, polyesters, polyamides, and combinations thereof. Highly preferred surface and core moieties include cross-linked PEG moieties, polyamine moieties, polyvinylamine moieties, and polyol moieties.

A preferred hydrophobic polymer to be used for production of beads of the composition of the invention is PS-DVB (polystyrene divinylbenzene). PS-DVB has been widely used for solid-phase peptide synthesis (SPPS), and has more recently demonstrated utility for the polymer-supported preparation of particular organic molecules (Adams et al. (1998) J. Org. Chem. 63:3706-3716). When prepared properly (Grøtli et al. (2000) J. Combi. Chem. 2:108-119), PS-DVB solid phase materials display excellent properties for chemical synthesis such as high loading, reasonable swelling in organic solvents and physical stability.

Linkers

The above-mentioned ligand is covalently immobilized to a solid phase material, possibly through a linker. In preferred embodiments, the ligand is covalently attached to a linker which is covalently attached to the polymer matrix. General techniques for linking of affinity ligands to solid phase materials can be found in Hermanson, Krishna Mallia and Smith, Immobilized Affinity Ligand Techniques”, Academic Press, 1992.

It should be understood that the linker should provide a suitable mobility of the ligand, but should not as such participate in the binding of the ligand to the antibody of interest. In fact, the binding of the immobilised ligand should be similar to the binding of the non-immobilised ligand.

Linkers are used for linking the ligand to a solid phase material such as e.g. a polymer matrix or an inorganic support. Preferably, the linker forms a strong and durable bond between the ligand and the solid phase material. This is particularly important, when the solid phase material of the present invention is to be used for repeated purification of antibodies.

However, in one embodiment of the present invention, linkers can be selectively cleavable. This can be useful when the solid phase material is to be used for analytical purposes.

Amino acids and polypeptides are examples of typical linkers. Other possible linkers include carbohydrates and nucleic acids.

In one embodiment, the linker residue L attached to the polymer matrix is cleavable by acids, bases, temperature, light, or by contact with a chemical reagent. In particular, the linker attached to the polymer matrix can be (3-formylindol-1-yl)acetic acid, 2,4-dimethoxy-4′-hydroxy-benzophenone, HMPA, HMPB, HMPPA, Rink acid, Rink amide, Knorr linker, PAL linker, DCHD linker, Wang linker and Trityl linker.

The ligand can be associated with the solid phase material through a linker having a length of preferably less than 50 Å, such as a length of from 3 to 30 Å, for example a length of from 3 to 20 Å, such as a length of from 3 to 10 Å.

Preferably, the linker is attached to the ligand via a carboxylic acid group, or an amino group, in particular via a carboxylic acid group.

The linker may also comprise a plurality of covalently linked subunits, e.g. such that the subunits are selected from identical and non-identical linker subunits. In one variant, the linker is flexible and comprises from 3 to preferably less than 50 identical or non-identical, covalently linked subunits.

In one embodiment of present invention, the linker L is selected from the group consisting of glycine, alanine, 3-aminopropionic acid, 4-aminobutanoic acid, and HMBA.

In embodiment of present invention, the linker can be selected from alkanes, such as linear alkanes, such as linear alkanes with 2-12 carbon atoms, monodisperse polyethyleneglycol (PEG), such as PEG with 2-20 repeat units, and peptides, such as peptides comprising 1-20 linked amino acids.

The linker can also be selected from the group consisting of polydispersed polyethylene glycol; monodispersed polyethylene glycol, such as triethylene glycol, tetraethylene glycol, pentaethylene glycol, hexaethylene glycol, heptaethylene glycol; an amino acid; a dipeptide; a tripeptide; a tetrapeptide; a pentapeptide; a hexapeptide; a heptapeptide; octapeptide; a nonapeptide; a decapeptide, a polyalanine; a polyglycine, including any combination thereof.

The solid phase material is most often presented in the form of beads, e.g. a particulate material having an average diameter of in the range of 0.1-1000 μm, or in the form of sticks, membranes, pellets, monoliths, etc.

Solution or Suspension

The expression “solution or suspension” herein is intended to mean a solid mass or/and liquid mass, comprising the Growth Hormone polypeptide, in particular a human Growth Hormone polypeptide. The expression “solution or suspension” is in particular meant to refer to a “large” volume or mass, i.e. referring to volumes and masses known from large-scale and industrial-scale processes.

The “suspension or solution of the Growth Hormone polypeptide typically originates from for e.g. a cell culture, a microbial process, a cloned animal (e.g. cows, pigs, sheep, goats, and fish) or insect, or the like, in particular from a cell culture or an industrial-scale production process. Alternatively, the suspension or solution of the Growth Hormone polypeptide may be derived from blood plasma, or the like.

The suspension or solution of the Growth Hormone polypeptide is typically obtained after lysing of cells in a particular cell culture or directly from cell culture fluid. The suspension or solution containing the Growth Hormone polypeptide can be subsequently adjusted by changing pH, ionic strength, or by chelation of divalent metal ions, etc., if desirable or beneficial.

In one embodiment of present invention, the suspension or solution containing the Growth Hormone polypeptide is obtained directly from a preceding purification step, or from a preceding purification step with subsequent adjustment of pH, ionic strength, chelation of divalent metal ions, etc., if desirable or beneficial.

Ligands

When used herein, the term “ligand” means a molecule which can bind a macromolecule (Ref: Physical Biochemistry: Applications to Biochemistry and Moleculare Biology, by David Freifelder. 2^{nd} edition W.H. Freemann and co. NY 1982. p 654) or a target compound, which in the present context, can be an antibody, in particular, human antibodies, such as human immunoglobulin (IgG) or more particularly recombinant IgG. The term “ligand” in present context more specifically refers to a “non-protein ligand”.

Preferably, the ligands in context of the present invention should bind to the antibody in question at least in a substantially specific manner (“specific binding”).

The expression “one or more ligands” refers to the fact that the solid phase material may have more than one type of ligand immobilized thereto. This being said, immobilization of a single type of ligands (“a ligand”) will typically involve the immobilization of a plurality/multitude of species of identical ligands.

In the present context, “specific binding” refers to the property of a ligand to bind to an antibody (i.e. the binding partner), preferentially such that the relative mass of bound antibody, is at least two-fold, such as 50-fold, for example 100-fold, such as 1000-fold, or more, greater than the relative mass of other bound species than the antibody. By relative mass of bound compound is meant the relative mass of bound specific binder=(mass specific bound/total compound bound)/(mass of bound non-specific/total compound bound).

Antibody

The terms “antibody”, “monoclonal antibody” and “polyclonal antibody” are defined and discussed below.

As used herein, the term “antibody” means an immunoglobulin, whether natural or wholly or partially synthetically produced. All fragments and derivatives thereof which maintain specific binding ability are also included in the term. Typical fragments are Fc, F(ab), heavy chain, and light chain. The term also covers any polypeptide having a binding domain which is homologous or largely homologous, such as at least 95% identical when comparing the amino acid sequence, to an immunoglobulin binding domain. These polypeptides may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. Derivatives of the IgG class, however, are preferred in one embodiment of the present invention.

Antibodies have one or more copies of a Y-shaped unit, composed of four polypeptide chains. Each Y contains two identical copies of a “heavy” chain, and two identical copies of a “light chain”, named as such by their relative molecular weights.

Antibodies can be divided into five classes: IgG, IgM, IgA, IgD and IgE, based on the number of Y units and the type of heavy chain. The heavy chain determines the subclass of each antibody. Heavy chains of IgG, IgM, IgA, IgD, and IgE are known as gamma, mu, alpha, delta, and epsilon, respectively. The light chains of any antibody can be classified as either a kappa (κ) or lambda (λ) type (a description of molecular characteristics of the polypeptide).

For pharmaceutical applications, the most commonly used antibody is IgG which can be cleaved into three parts, two F(ab) regions and one Fc region, by the proteolytic enzyme papain, or into two parts, one F(ab′)₂ and one Fc region by the proteolytic enzyme pepsin.

The F(ab) regions comprise the “arms” of the antibody, which are critical for antigen binding. The Fc region comprises the “tail” of the antibody and plays a role in immune response, as well as serving as a useful “handle” for manipulating the antibody during some immunochemical procedures. The number of F(ab) regions on the antibody, corresponds with its subclass, and determines the “valency” of the antibody (loosely stated, the number of “arms” with which the antibody may bind its antigen).

The term “antibody fragment” refers to any derivative of an antibody which is less than full-length. Preferably, the antibody fragment retains at least a significant portion of the specific binding ability of the full-length antibody. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

“Single-chain Fvs” (scFvs) are recombinant antibody fragments consisting of only the variable light chain (V_{L}) and variable heavy chain (V_{H}) covalently connected to one another by a polypeptide linker. Either V_{L} or V_{H} may be the amino-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility. “Diabodies” are dimeric scFvs. The components of diabodies typically have shorter peptide linkers than most scFvs and they show a preference for associating as dimers. An “Fv” fragment is an antibody fragment which consists of one V_{H} and one V_{L} domain held together by non-covalent interactions. The term “dsFv” is used herein to refer to an Fv with an engineered intermolecular disulfide bond to stabilize the V_{H}-V_{L} pair. A “F(ab′)₂” fragment is an antibody fragment essentially equivalent to that obtained from immunoglobulins (typically IgG) by digestion with an enzyme pepsin at pH 4.0-4.5. The fragment may be recombinantly produced. A “Fab′” fragment is an antibody fragment essentially equivalent to that obtained by reduction of the disulfide bridge or bridges joining the two heavy chain pieces in the F(ab′)₂ fragment. The Fab′ fragment may be recombinantly produced. A “Fab” fragment is an antibody fragment essentially equivalent to that obtained by digestion of immunoglobulins (typically IgG) with the enzyme papain. The Fab fragment may be recombinantly produced. The heavy chain segment of the Fab fragment is the Fd piece. A “Fc” region is a constant region of a particular class of antibody.

The bonding between antigens and antibodies is dependent on hydrogen bonds, hydrophobic bonds, electrostatic forces, and van der Waals forces. These are all bonds of a weak, non-covalent nature, yet some associations between an antigen and an antibody can be quite strong. Accordingly, the affinity constant for antibody-antigen binding can span a wide range, extending from below 10⁵ mol⁻¹ to more than 10¹² mol⁻¹. Affinity constants are affected by temperature, pH and solvent. Apart from an affinity of an antibody for a ligand, the overall stability of an antibody-ligand complex is also determined by the valency of the antigen and antibody and the structural arrangement of the interacting parts.

Accurate affinity constants can only be determined for monoclonal antibodies which are genetically identical molecules recognising one single epitope on the antigen whereas for polyclonal antibodies a broad distribution of affinities may contribute to an apparent affinity constant. The apparent affinity constant may also be caused by the fact that polyclonal antibodies may recognise more than one single epitope on the same antigen. Since antibodies normally harbour more than one binding domain per molecule multiple, co-operative bondings take place between antibodies and their antigens; this effect is termed avidity. As monoclonal antibodies react with only one single epitope on the antigen they are more vulnerable to the loss of epitope through chemical treatment of the antigen than polyclonal antibodies. This can be offset by pooling two or more monoclonal antibodies to the same antigen.

Monoclonal antibodies can be raised by fusion of B lymphocytes with immortal cell cultures to produce hybridomas. Hybridomas will produce many copies of the exact same antibody—an essential feature in the development of antibodies for therapeutic or diagnostic applications.

Preparation of Affinity Resins

The affinity resins can in principle be prepared in two fundamentally different ways, namely (i) by synthesizing the ligand in free form and subsequently immobilizing the ligand to the solid phase material directly or via a linker (see above), or (ii) by functionalizing the solid phase material and thereafter sequentially synthesizing the ligand(s). With respect to the first variant, immobilization techniques are readily available in the art, e.g. in Hermanson et al. (see above). With respect to the second variant, techniques are also readily available, e.g. the techniques known in the art of solid phase peptide synthesis and derived techniques (Fields, G. B. et al. (1992) Principles and practice of solid-phase peptide synthesis. In Synthetic Peptides: A User's Guide (Grant, G. A., ed.), pp. 77-183, W. H. Freeman; Fields, G. B., ed. (1997) Solid-phase peptide synthesis. Methods in Enzymology 289 and Dorwald, F. Z. Organic synthesis on solid phase—supports, linkers, reactions; Wiley-VCH: Weinheim, 2000).

In a first step of the process, the solution or suspension containing the antibody is contacted with an affinity resin under conditions which facilitate binding of a portion of said antibody to said affinity resin. The aim is to facilitate binding of a relevant portion of said antibody to said affinity resin.

By the term “contacting the solution or suspension containing antibody/antibody” in connection with step (a) means that

By the term “portion” in connection with step (a) is meant at least 30% (i.e. 30-100%) of the mass of the antibody present in the solution or suspension containing the antibody. It should be understood that it in most instances is desirable to bind far more than 30% of the mass of the antibody, e.g. at least 50%, or at least 70%, or a predominant portion. By the term “predominant portion” is meant at least 90% of the mass of the antibody present in the solution or suspension. Preferably an even higher portion becomes bound to the affinity resin, e.g. at least 95% of the mass, or at least 98% of the mass, or at least 99% of the mass, or even substantially all of the mass of the antibody present in the solution or suspension containing the antibody.

The solution or suspension containing the antibody typically originates from a production process/processes, such as a cell culture, industrial-scale/industrial process, a microbial process, a cloned animal (e.g. cows, pigs, sheep, goats, and fish) or insect, or the like, in particular from a cell culture. Alternatively, the solution or suspension of the antibody may be derived from blood plasma, or the like.

The most common arrangement of the affinity resin is in a column format. Arrangement in a batch container is of course also possible.

The solution or suspension is typically obtained directly from cell culture fluid or from cell culture fluid with subsequent adjustment of pH, ionic strength, chelation of divalent metal ions, etc., if desirable or beneficial. In another embodiment, the solution or suspension is obtained directly from a preceding purification step, or from a preceding purification step with subsequent adjustment of pH, ionic strength, chelation of divalent metal ions, etc., if desirable or beneficial.

The contacting of the antibody present in the solution or suspension is typically conducted according to conventional protocols, i.e. the concentration, temperature, ionic strength, etc. and the affinity resin may be washed and equilibrated before application as usual.

The load of antibody is typically at least 5 g per litre of affinity resin, such as in the range of 1-30 g, e.g. 3-15 g, antibody per litre of affinity resin in wet form, and the solution or suspension containing the antibody is typically loaded at a flow of 1-50 column volumes per hour (CV/h), such as 25-35 CV/h.

The pH of the solution or suspension before and upon application to the affinity resin appears to play a relevant role for the formation of contaminants, e.g. in the form of dimers and degradation products of the antibody. Thus, it is preferred that the solution or suspension is in liquid form and has a pH in the range of 3.0-10.0, such as in the range of 3.0-7.0, or 6.5-10.0, upon application to the affinity resin. In some interesting embodiments, the solution or suspension containing the antibody has a pH of in the range of 4.0-7.0, or in the range of 7.0-9.0, or in the range of 4.5-8.5. A preferred pH range would be 6.0-8.0.

The temperature of the solution or suspension containing the antibody is typically 10-30° C., such as around 15-25° C.

The temperature of the affinity resin with the bound antibody is typically 10-30° C., such as around 15-25° C., e.g. kept within a specified range by using a cooling and/or heating jacket and solutions of controlled temperature.

Step (b)—Washing Step (Optional)

After binding of the antibody to the affinity resins, a washing step (b) is typically conducted in order to remove proteins which are bound unspecific to the affinity resin. By this step, the remaining (bound) fraction of the antibody on the affinity resin will have a much lower abundance of contaminants.

This washing step (b) is preferably conducted with a washing buffer having a pH in the range of 2.0-6.9. In some interesting embodiments, the washing buffer has a pH in the range of 6.0-10.0, such as in the range of 6.0-7.0, or 6.5-10.0, upon application to the affinity resin. In some interesting embodiments, the washing buffer has a pH of in the range of 6.0-7.0, or in the range of 7.0-9.0, or in the range of 3.0-5.0.

The washing step (b) is typically conducted at a flow of 1-50 column volumes per hour.

The washing buffer is typically an aqueous solution comprising a buffering agent, typically a buffering agent comprising at least one component selected from the groups consisting of acids and salts of MES, PIPES, ACES, BES, TES, HEPES, TRIS, BISTRIS, triethanolamine, histidine, imidazole, glycine, glycylglycine, glycinamide, phosphoric acid, acetic acid (e.g. sodium acetate), lactic acid, glutaric acid, citric acid, tartaric acid, malic acid, maleic acid, and succinic acid. It should be understood that the buffering agent may comprise a mixture of two or more components, wherein the mixture is able to provide a pH value in the specified range. As examples can be mentioned acetic acid and sodium acetate, etc.

In addition to a buffering agent, the washing buffer may also contain non-ionic detergents such as NP40, Triton-X100, Tween-80, or other additives such as caprylic acid.

In addition to a buffering agent, the washing buffer may also contain ionic strength increasing agents that do not change the pH of the buffer, such as sodium chloride, sodium sulphate and the like.

The elution buffer can contain 5-30% v/v glycerol or propylene glycol, in combination with either of the above mentioned buffers.

In one currently preferred embodiment, step (b) involves at least one washing buffer comprising phosphoric acid buffer.

It should be understood that the washing step (b) may be conducted by using one, two or several different washing buffers, or by the application of a gradient washing buffer.

It should also be noted that the washing step and the elution step need not to be discrete steps, but may be combined, in particular if a gradient elution buffer is utilised in the elution step.

Step (c)—Elution Step

After the washing step(s) (c), the affinity resin is eluted with an elution buffer, and a purified antibody is collected as an eluate.

A great deal of variability is possible for the elution step (c).

The type of elution is not particularly critical, thus, it is, e.g., possible to elute with an elution buffer comprising a stepwise decreasing gradient of salts, elute with a linear decreasing gradient of the salts (or a gradient-hold-gradient profile, or other variants), or to use a pH gradient, or to use a temperature gradient, or a combination of the before-mentioned.

The conductivity of the final elution buffer is preferably higher than the conductivity of the composition of solution or suspension comprising the antibody in step (a).

In most instances, the elution buffer in step (c) typically has a lower pH than in step (a) and (b). However, the elution buffer in step (c) may also have a pH higher than in step (a) and (b).

Also preferred are the embodiments where the elution buffer in step (c) has a pH between 3.0 and 5.0.

In one embodiment of present invention, the elution buffer comprises 10 mM formic acid.

In one embodiment of present invention, the elution buffer has a pH between 3.0 and 5.0 and comprises an ionic strength increasing agent such as sodium chloride in a preferred concentration from 10-500 mM.

The elution step (c) is typically conducted at a flow of 1-50 column volumes per hour.

Typically, the process of the present invention is capable reducing the content of other proteins with at least 50%, however more preferably, and also realistically, the reduction is at least 60%, such as at least 70% or even at least 80% or at least 85%.

Usually, the affinity resin can be regenerated for the purpose of subsequent use by a sequence of steps.

It should be noted that the washing step and the elution step need not to be discrete steps, but may be combined, in particular if a gradient elution buffer is utilised in the elution step.

Although not limited thereto, the process of the present invention is particularly feasible for “industrial-scale” (or “large-scale”) solutions or suspensions containing antibody. By the term “industrial-scale” is typically meant methods wherein the volume of liquid antibody compositions is at least 10 L, such as at least 50 L, e.g. at least 500 L, or at least 5000 L, or where the weight of the product is at least 10 g (dry matter), such as at least 100 g, e.g. at least 500 g, e.g. 1-15,000 g.

Novel Affinity Resins

It is believed that some of the most interesting affinity resins described herein are novel as such. Hence, the present invention also provides novel affinity resins comprising a solid phase material having covalently immobilized there to one or more ligands, i.e. the ligands described hereinabove.

The present invention also provides novel affinity resins comprising a solid phase material having covalently immobilized there to one or more ligands, i.e. the ligands described hereinabove.

One embodiment of present invention provides a process for the purification of an antibody, said process comprising the steps of:

(a) contacting a solution or suspension containing antibody with an affinity resin under conditions which facilitate binding of a portion of the antibody to said affinity resin;

(b) optionally washing said affinity resin containing bound antibody with a washing buffer; and

(c) eluting said affinity resin containing bound antibody with an elution buffer, and collecting a purified antibody as an eluate;

wherein said affinity resin is a solid phase material having covalently immobilized thereto one or more ligands of the general formula (I),

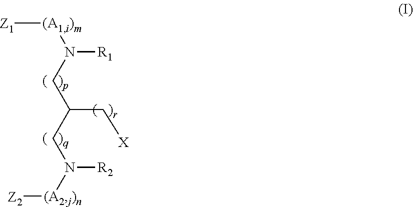

wherein i=1,2, . . . , m,

wherein j=1,2, . . . , n,

wherein n and m are independently an integer in the range of 0-3, with the proviso that the sum n+m is in the range of 1-4,

wherein p, q, and r are independently an integer in the range of 0-6,

A11, . . . , A1m, and A21, . . . , A2n are independently selected from α-amino acid moieties, β-amino acid moieties, α-amino sulphonic acid moieties, and β-amino sulphonic acid moieties,

Z1 and Z2 are independently selected from hydrogen, C₁₋₆ alkyl, carboxylic acid moieties (Z—C(═O)—) and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl,

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl and Hal is a halogen; and

the total molecular weight of said ligand (excluding “X” and any linker) being 200-2000 g/mol.

One embodiment of present invention provides a process of purification of antibody wherein A11, . . . , A1m and A21, . . . , A2n independently are selected from glycine, L-proline, L-arginine, L-tyrosine, D-glutamine, D-tyrosine, L-valine, L-cysteine, L-histidine, and L-leucine;

One embodiment of present invention provides a process of purification of antibody wherein Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl;

R1 and R2 are independently selected from hydrogen and C₁₋₆-alkyl;

m is 0 or 1, n is 0 or 1, (p,q) is (0,1), (0,2), (0,3), (0,4), (1,0), (2,0), (3,0), or (4,0), and r is 0.

One embodiment of present invention provides a process of purification of antibody, wherein the ligand has the general formula (II),

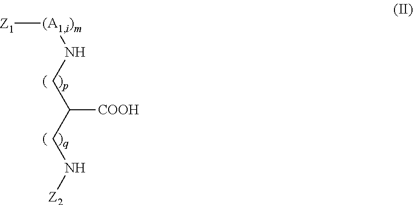

One embodiment of present invention provides a process of purification of antibody, wherein

p and q are independently an integer in the range of 0-6,

wherein i=1,2, . . . , m, and m is an integer in the range of 1-4, such as 1-3, e.g. 1-2;

A11, . . . , A1m independently are selected from glycine, L-proline, L-arginine, L-tyrosine, D-glutamine, D-tyrosine, and L-valine;

Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl;

5. The process according to claim 4 wherein the ligand is selected from (1)-(12):

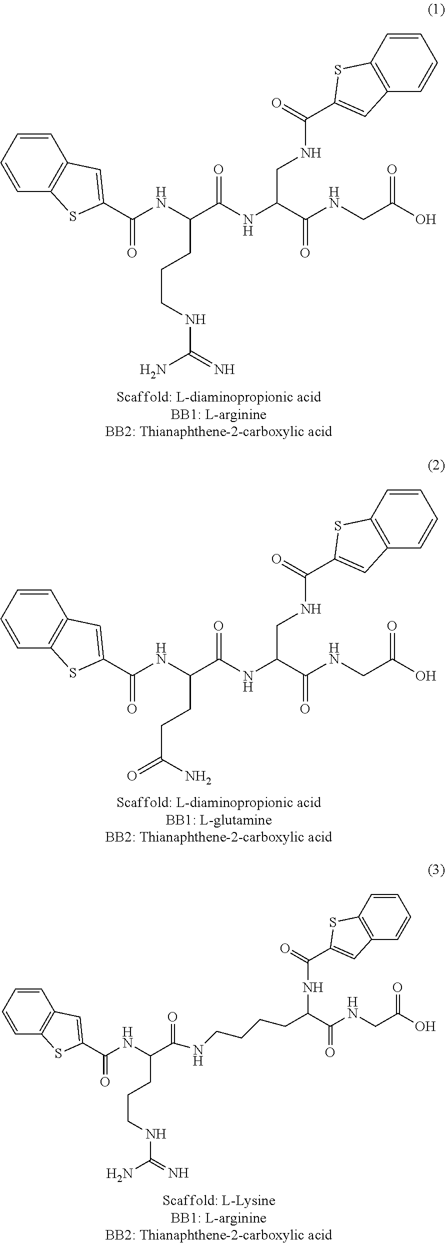

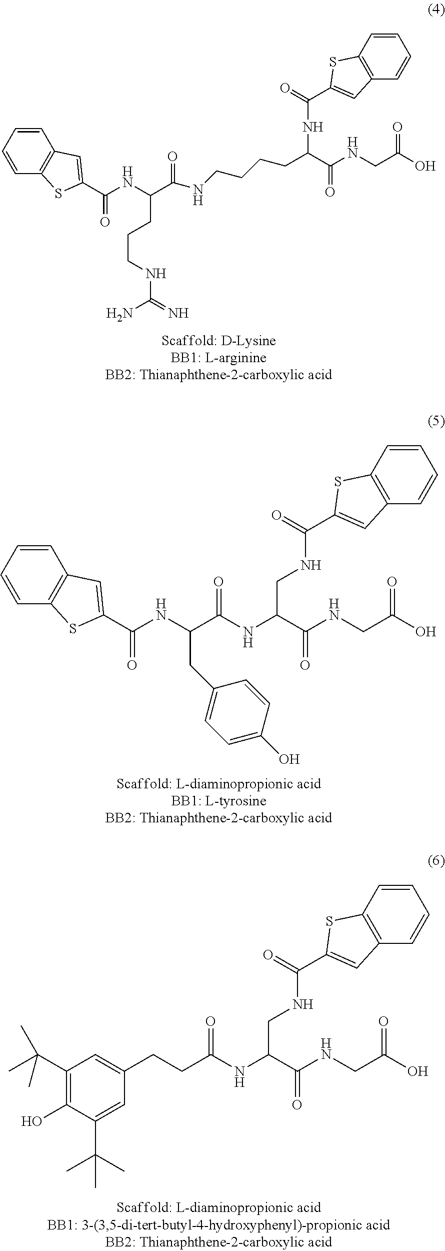

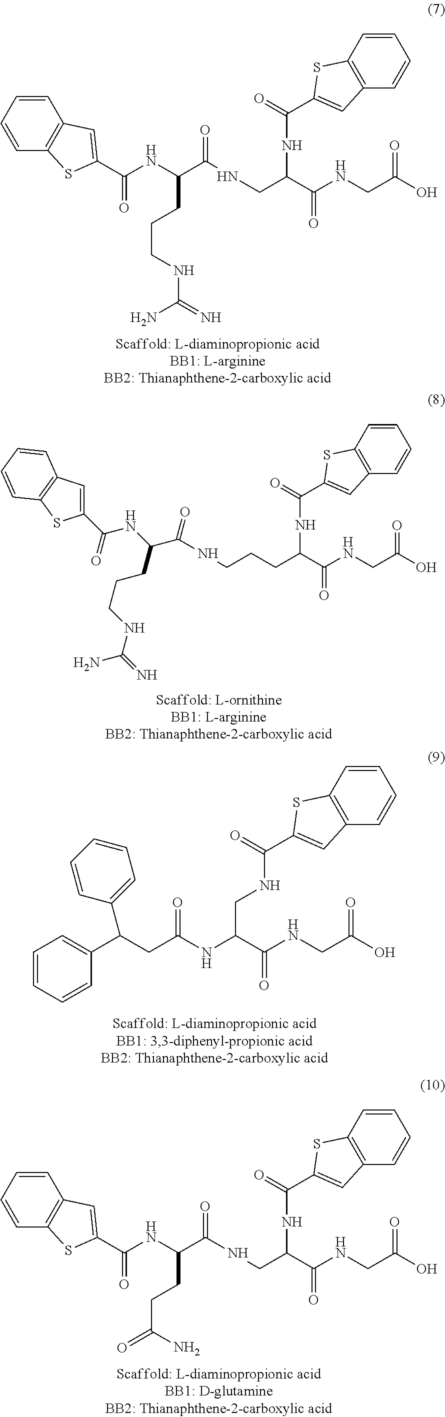

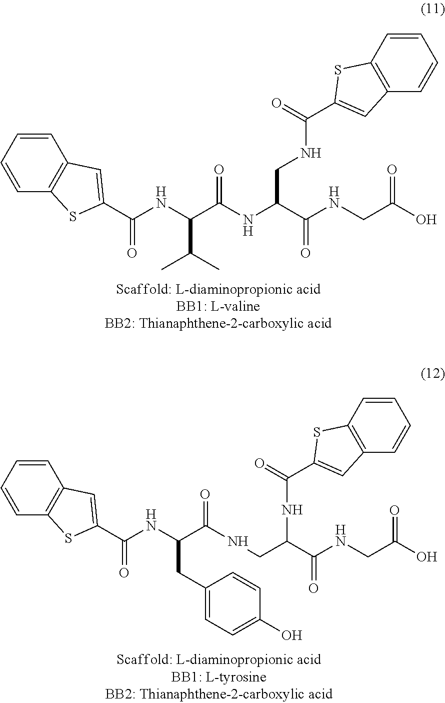

One embodiment of present invention provides a process of purification of antibody wherein ligand has the general formula (III)

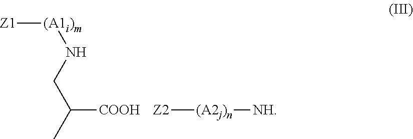

7. An affinity resin comprising a solid phase material having covalently immobilized thereto one or more ligands of the formula

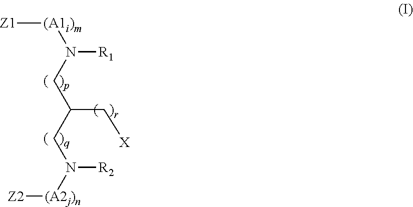

wherein i=1,2, . . . , m,

wherein j=1,2, . . . , n,

wherein n and m are independently an integer in the range of 0-3, with the proviso that the sum n+m is in the range of 1-4,

wherein p, q, and r are independently an integer in the range of 0-6,

A11, . . . , A1m, and A21, . . . , A2n, are independently selected from α-amino acid moieties, β-amino acid moieties, α-amino sulphonic acid moieties, and β-amino sulphonic acid moieties,

Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl,

R1 and R2 are independently selected from hydrogen and C₁₋₆-alkyl;

X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl and Hal is a halogen; and

the total molecular weight of said ligand (excluding “X” and any linker) being 200-2000 g/mol.

One embodiment of present invention provides for an affinity resin, wherein the ligand has the general formula (II),

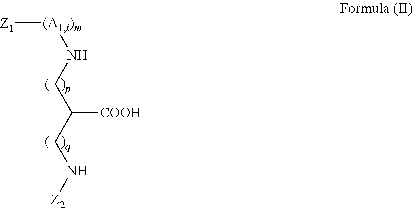

One embodiment of present invention provides for an affinity resin, wherein the ligand has the general formula (III)

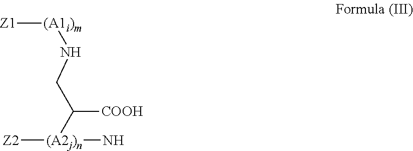

One embodiment of present invention provides for an affinity ligand selected from the group consisting of ligands (1)-(12) illustrated as above.

In one embodiments of present invention, the ligands are as specified hereinabove for general formulae (I), (II) and (III) and further in accordance with the various embodiments, in particular those embodiments of the general formulae (II) and (III). The currently most interesting ligands are ligands Nos. (1)-(12) illustrated above.

The novel affinity resins are particularly useful in the purification and/or isolation of biomolecules, such as proteins, in particular antibodies. The affinity ligands are specific binding partners for antibodies and can isolate said antibodies from closely related proteins.

In one embodiment of present invention, the ligand is immobilized to the surface of a sensor or an array plate (the “solid phase material”) and is used to detect and/or quantify antibodies in a biological sample.

When used herein, the term “biological sample” includes natural samples or samples obtained from industrial processes, e.g. recombinant processes, and include “body fluid”, i.e. any liquid substance extracted, excreted, or secreted from an organism or tissue of an organism. A body fluid need not necessarily contain cells. Body fluids of relevance to the present invention include, but are not limited to, whole blood, serum, urine, plasma, cerebral spinal fluid, tears, milk, sinovial fluid, and amniotic fluid.

In one embodiment of present invention, a plurality of ligands are immobilized to the surface of an array plate (the “solid phase material”) and arranged in a plurality of spots, with each spot representing one ligand. Such a functionalized array can be used to detect the presence of antibodies in a solution. Such an array can be used for diagnostic applications to detect the presence of antibodies in a biological sample.

In one embodiment of present invention, a plurality of ligands are immobilized to the binding surface of a cantilever sensor (the “solid phase material”) for detection and optionally quantification of antibodies. A plurality of affinity ligands can be immobilized to a plurality of cantilevers with each cantilever representing one ligand. Such a functionalized array can be used to detect the presence of various antibodies in a solution. Such a multi-sensor can be used for diagnostic applications to detect the presence of certain antibodies in a biological sample.

Furthermore, it is believed that some of the ligands are novel as such.

Hence, the invention further provides affinity ligands as specified above with general formulae (I), (II) and (III), in particular those selected from the group consisting of ligands (1)-(12).

### EXAMPLES

### Example 1

### Selection of Building Blocks for Combinatorial Library Using PCA

The combinatorial library was designed by employing two parallel approaches. First a virtual combinatorial library comprising 229,957 members was generated. For each of the virtual library members a set of 118 physico-chemical descriptors was calculated according to the procedure of Cruciani et al [Ref. Cruciani, C., et al., Molecular fields in quantitative structure-permeation relationships: the VolSurf approach. Journal of Molecular Structure-Theochem, 2000. 503 (1-2): p. 17-30; Cruciani, G., M. Pastor, and W. Guba, VolSurf: a new tool for the pharmacokinetic optimization of lead compounds. European Journal of Pharmaceutical Sciences, 2000. 11: p. S29-S39]. Multivariate statistics were used to analyse the resulting model. In order to simplify the model the 118 descriptors were projected onto two principal components.

The combinatorial library was then optimized for chemical diversity by employing design of experiments to rationally select the 294 structures that span the largest possible part of the thirteen-dimensional chemical space. The frequency of the building blocks in these ligands was used to select over-represented building blocks, which were all included in the combinatorial library to be synthesized.

All selected building blocks were subsequently tested for coupling yield in the lab, and building blocks found to exhibit low coupling yields or found to form by-products were discarded. The outcome was a library consisting of 770 structures.

### Example 2

### Synthesis of Combinatorial Library

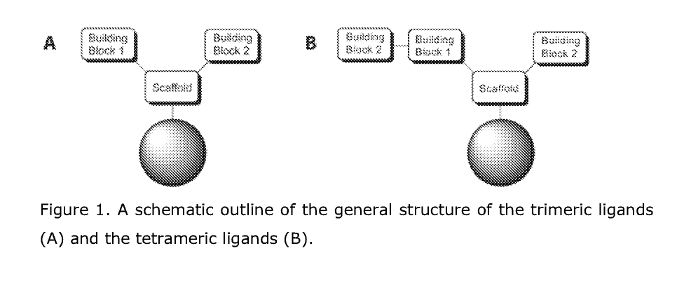

The general structure of the combinatorial library of affinity ligands aimed at binding to Fc fragment is shown in FIG. 1. The Scaffolds were selected from aliphatic di-amino-carboxylic acids, Building blocks 1 and Building blocks 2 were selected independently from natural amino acids, unnatural amino acids, and carboxylic acids.

FIG. 1. A schematic outline of the general structure of the trimeric ligands (A) and the tetrameric ligands (B).

The combinatorial library was synthesized by employing one-bead-one-compound split and mix solid phase synthesis. The 770 different ligands were synthesized on approximately 20,000 beads optically encoded amino-functional polyethyleneglycol-acrylamide (PEGA) beads. To keep track of all compounds throughout the synthesis, the encoded bead technology was used for reading the optically encoded beads used for the synthesis [WO 2005/061094, WO 2005/062018. The building blocks used for the synthesis are provided in Table I.

TABLE I
A complete list of the building blocks used for the library synthesis. The
building blocks are provided with protection groups as used in the synthesis. For
the coupling to the scaffold, the Fmoc-protected amines were reacted first.

| Scaffold | | Building Block 1 | | Building Block 2 | |
|---|---|---|---|---|---|
| 1 | 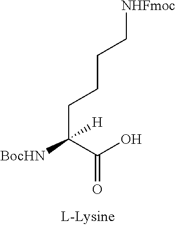 | 1 | 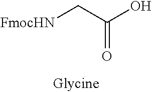 | 1 | 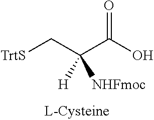 |
| 2 | 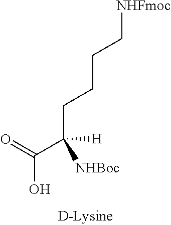 | 2 | 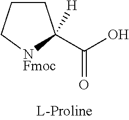 | 2 | 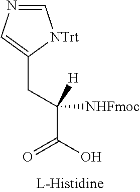 |
| 3 | 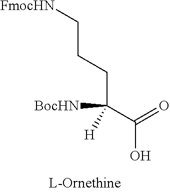 | 3 | 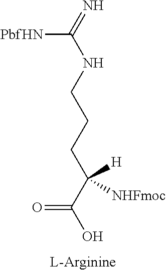 | 3 | 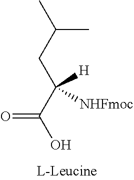 |
| 4 | 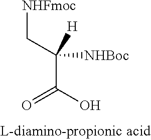 | 4 | 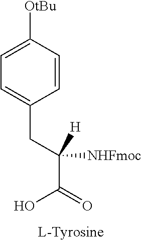 | 4 | 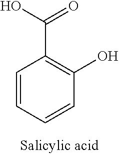 |
| 5 | 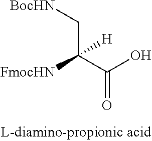 | 5 | 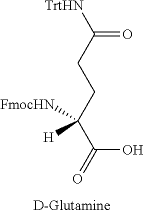 | 5 | 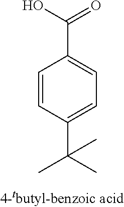 |
| | | 6 | 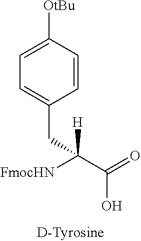 | 6 | 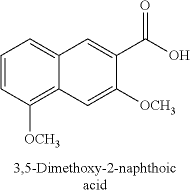 |
| | | 7 | 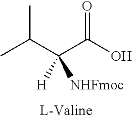 | 7 | 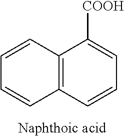 |
| | | 8 | 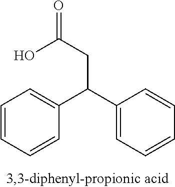 | 8 | 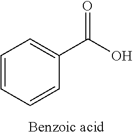 |
| | | 9 | 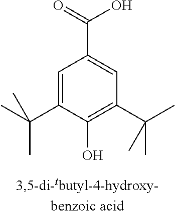 | 9 | 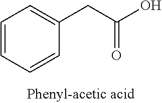 |
| | | 10 | 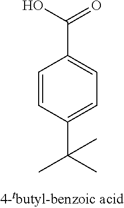 | 10 | 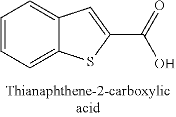 |
| | | 11 | 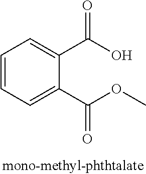 | 11 | 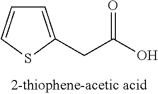 |
| | | 12 | 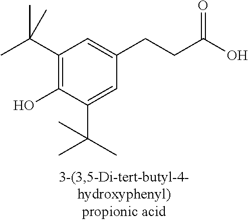 | | |
| | | 13 | 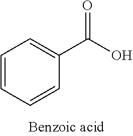 | | |
| | | 14 | 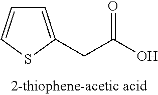 | | |

4.8 mL PEGA1900 encoded beads, were placed in a 10 mL syringe and washed 4 times with NMP (8 mL). The beads were then allowed to swell in the solvent (8 mL) for 45 minutes. The beads were then drained, and the Fmoc protecting group removed by treatment with 20% piperidine in NMP. The deprotection was confirmed by a positive Kaiser test. Subsequently the resin was divided equally into five 2 ml reaction syringes. The protected diamino scaffold (10 eq) were preactivated with TBTU (9.7 eq) and DIPEA (13.3 eq) for 5 min and then added to the appropriate reactors. The coupling was carried out under vigorous shaking for 45 min at rt. The resin was then drained and washed with NMP (8×, 2 ml; 2×5 min). From each reactor a few beads were removed and a Kaiser test was performed. In all cases a negative Kaiser test resulted. Following the Kaiser test the beads were washed with water (10×, 2 ml; 4×5 min) and the bead codes were then read on the bead decoder.

After code recording, the resin from the individual reactors was mixed and transferred to a 10 ml syringe, where they were washed with NMP (8×, 8 ml, 2×5 min+2×10 min). The Fmoc groups were cleaved as described previously. The resin was divided equally into 14×2 ml reaction syringes. Building blocks 1 (10 eq) were preactivated with TBTU (9.7 eq) and DIPEA (13.3 eq) for 5 min and then added to the appropriate reactors. The coupling was carried out under vigorous shaking for 45 min at rt for amino acids while the remaining building blocks were coupled for 4 h. Following the coupling, the resin was drained, washed with NMP (8×, 2 ml, 2×5 min), with water (10×, 2 ml, 2×10 min+2×5 min) and bead code read. The second building block was coupled in a like manner.

### Example 3

### Screening of Library and Identification of Binding Ligands

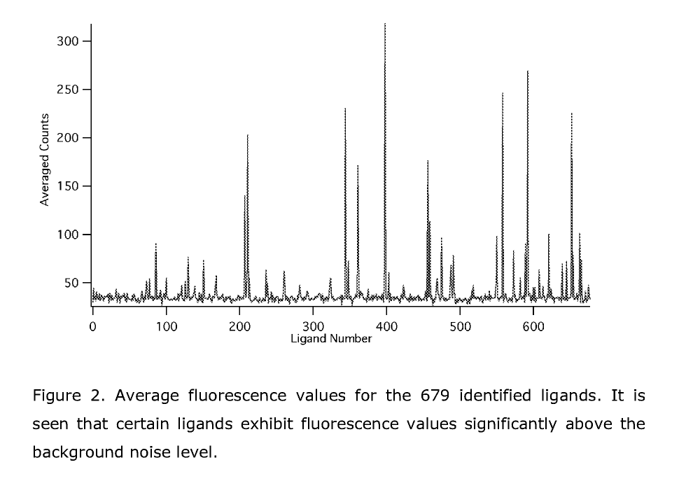

The combinatorial library was after the last synthesis step divided into 11 reactors. These were washed 10 times with freshly made PBS buffer pH 7.4. To 3.4 ml of Rohdamine X labelled Fc-fragment, was added 1.85 ml PBS buffer at pH 7.4 to yield a final protein concentration of 0.40 mg/ml. 400 μl (160 μg protein) was added to each reactor, and the library was incubated overnight (16 h). The following morning the reactors were washed 10 times with PBS buffer and 10 times with MilliQ water. The bead code and the fluorescence intensity of each bead was then recorded. Data was obtained for 679 of the 770 synthesized ligands. A plot of the average fluorescence for each of the 679 ligands is provided in FIG. 2.

FIG. 2. Average fluorescence values for the 679 identified ligands. It is seen that certain ligands exhibit fluorescence values significantly above the background noise level.

Based on the fluorescence and decoding data, we found that the 12 structures provided in Table II were the most promising affinity ligands based on their high average fluorescence values.

TABLE II
Schematic representation of the 12 ligands (bound to the solid phase),
which were found to be the most promising due to their high fluorescence
values, coupled to a solid phase material.

| | |
|---|---|
| 1 | 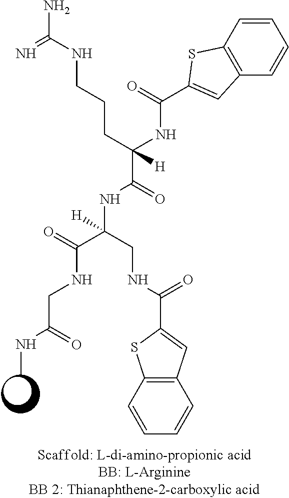 |
| 2 | 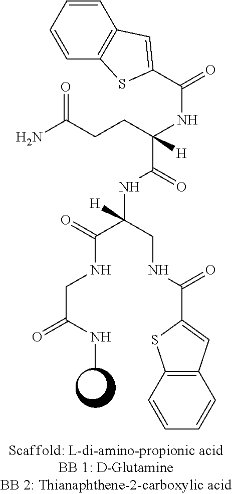 |
| 3 | 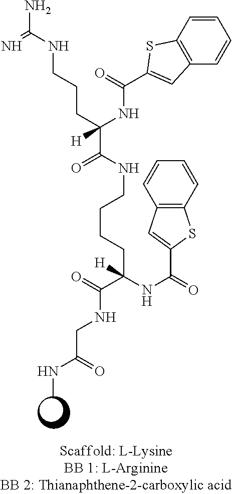 |
| 4 | 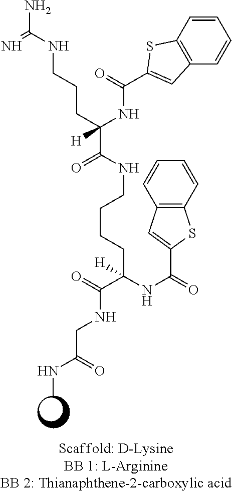 |
| 5 | 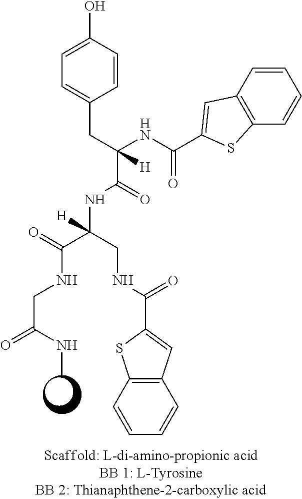 |
| 6 | 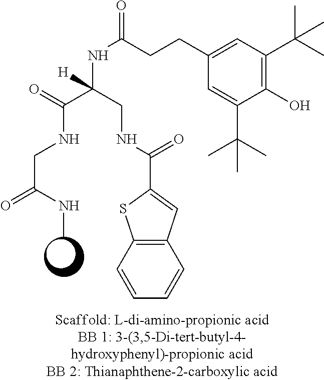 |
| 7 | 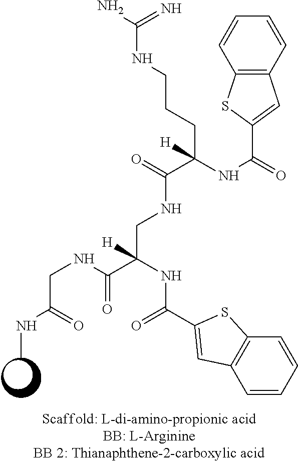 |
| 8 | 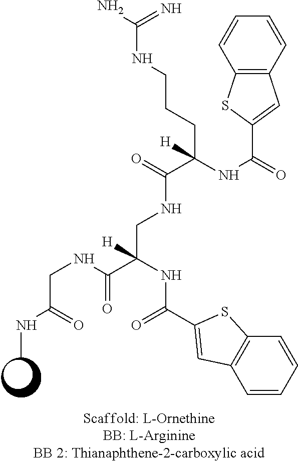 |
| 9 | 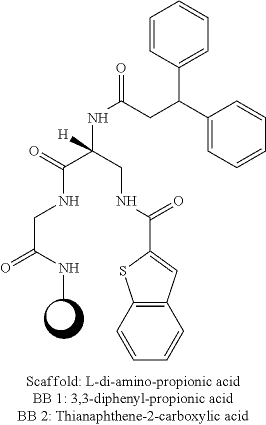 |
| 10 | 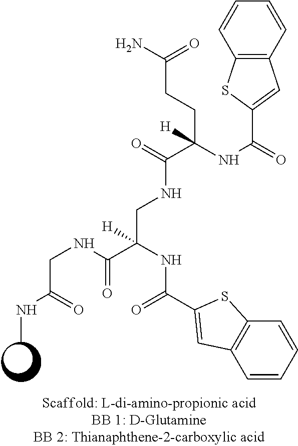 |
| 11 | 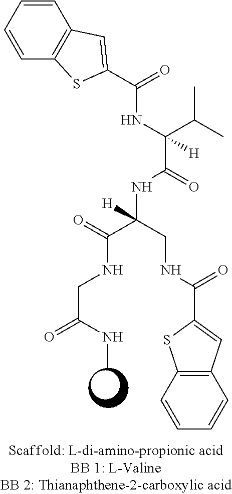 |
| 12 | 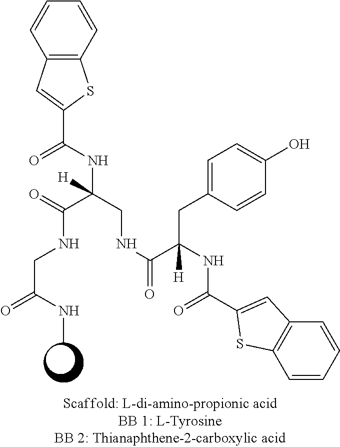 |

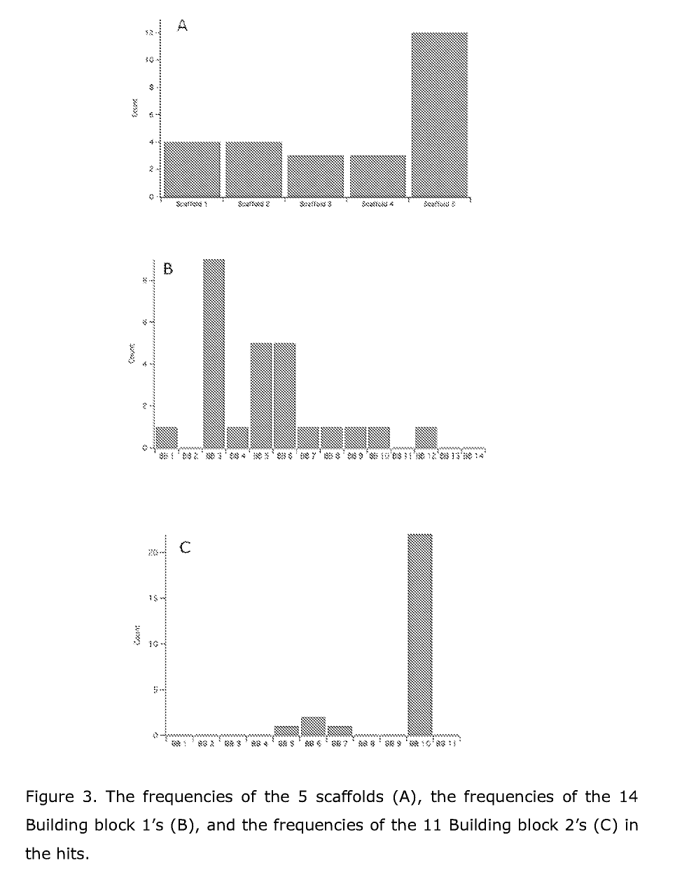

We classified the 26 ligands found to have an average fluorescence above 69 as “hits”. We determined the frequency of the Scaffolds and Building blocks 1 and 2 in the hits. The result is shown in FIG. 3.

FIG. 3. The frequencies of the 5 scaffolds (A), the frequencies of the 14 Building block 1's (B), and the frequencies of the 11 Building block 2's (C) in the hits.

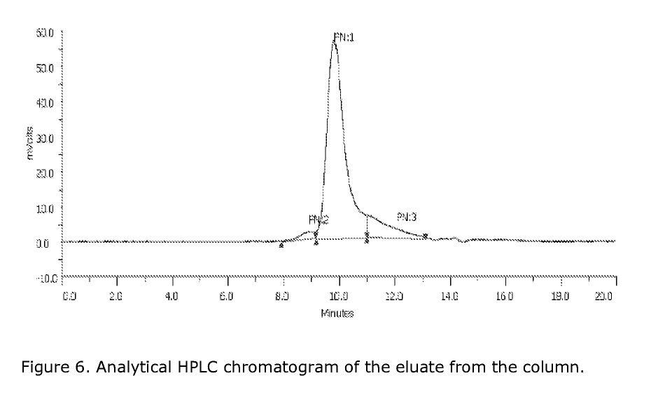

FIG. 6 shows that especially Scaffold no. 5 (L-di-aminopropionic acid), Building block 1 no. 3 (L-Arginine), Building block 1 no. 5 (L-Tyrosine), Building block 1 no. 6 (D-Glutamine), and Building block 2 no. 10 (thianapthene-2-carboxylic acid) are over-represented.

### Example 4

### Resynthesis of Ligand Hits and Coupling of Chromatography Resin

Ligand Synthesis

The ligands were synthesized on gram scale by solid phase using an HMBA linker and Fmoc chemistry. After cleavage from the resin with sodium hydroxide to provide the ligand acid, it was purified by HPLC.

Ligand Coupling to Amino Sepharose

The synthesized ligands were coupled to an amino-sepharose (10 μmol/ml, GE Healthcare) chromatographic resin. For each of the couplings 1.7 ml amino-sepharose was swollen in NMP over a period of 1 hour. The Ligand (3.5 eq) in NMP was preactivated with EDC (3 eq), HOAt (3 eq) and DIPEA (4 eq) at rt for 5 min then added to the resin. The reaction tube was then sealed and the reaction was allowed to proceed for 3 hours at 60° C. under shaking. After the coupling time had elapsed, the resin was washed 10 times with NMP, ethanol and water.

Ligand Coupling to Fractogel

Two 1.2 ml portions of amino Fractogel were placed in two reactors and washed thoroughly with NMP over 45 minutes. The Ligand (3.5 eq) in NMP was preactivated with EDC (3 eq), HOAt (3 eq) and DIPEA (4 eq) at rt for 5 min then added to the resin. The reaction tube was then sealed and the reaction was allowed to proceed for 3 hours at 60° C. under vigorous shaking. After the coupling time had elapsed, the resin was washed with NMP (10×, 8 ml) ethanol and water

### Example 5

### Purification of Antibody Using Affinity Ligands Bearing Fc-Binding Ligands

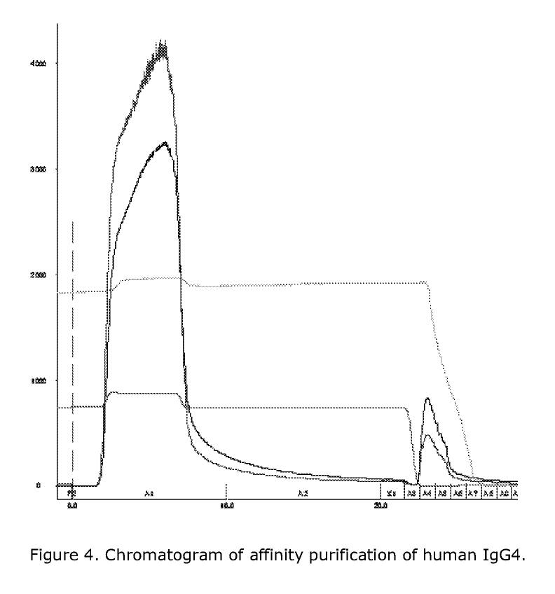

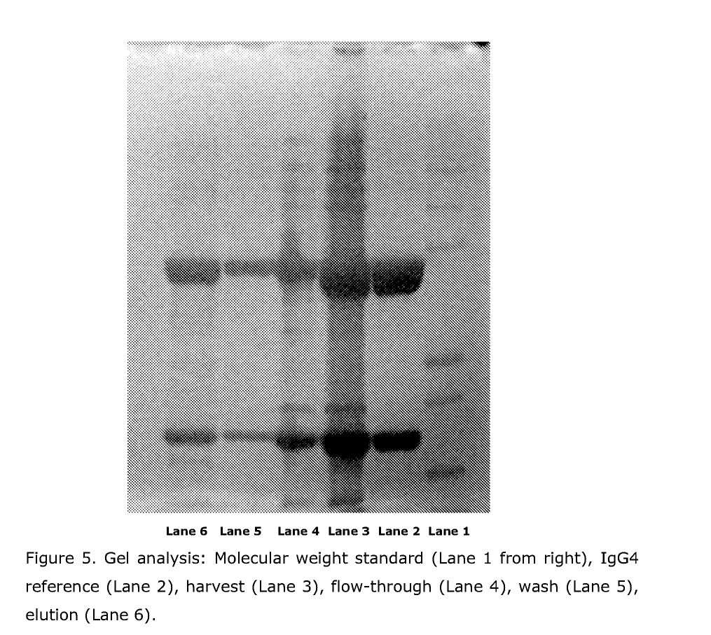

The resin was packed in a Tricorn 5/50 column (GE Healthcare) to a bed volume of 1 ml (5.1 cm bed height). The column was coupled to the ÄKTA100 Explorer system and equilibrated with 5 ml of equilibration buffer i.e. 50 mM NaP, pH 7, 0.1 M NaCl. The antibody feed was loaded to the column by the superloop followed by washing with 15 ml of wash buffer i.e. 50 mM NaP, pH 7, 0.1 M NaCl. Adsorbed antibody to the column was then eluted by 15 ml of elution buffer e.g. 10 mM Na-Formate, pH 3.6, 100 mM NaCl. After the elution step the column was regenerated using a cleaning in place step with 5 ml of CIP-solvent e.g. 1 M sodium hydroxide solution followed by a re-equilibration step with 10 ml of equilibration buffer i.e. 50 mM NaP, pH 7, 0.1 M NaCl (not shown in FIG. 1). The flow rate for the equilibration, load, wash and elution step was 0.33 ml/min (100 cm/h) and the flow rate for the CIP and re-equilibration step was 0.5 ml/min. A Typical chromatogram is shown in FIG. 4. Eluted fractions were adjusted to pH 7 by 0.5 M Na₂HPO₄ if necessary and then analysed for purity by SEC-HPLC (FIG. 6) and SDS-PAGE (FIG. 5). The antibody was obtained in approximately 85% purity.

1. A process for the purification of an antibody, said process comprising the steps of:
(a) contacting a solution or suspension containing antibody with an affinity resin under conditions which facilitate binding of a portion of the antibody to said affinity resin;
(b) optionally washing said affinity resin containing bound antibody with a washing buffer; and
(c) eluting said affinity resin containing bound antibody with an elution buffer, and collecting a purified antibody as an eluate;
wherein said affinity resin is a solid phase material having covalently immobilized thereto one or more ligands of the general formula (I), 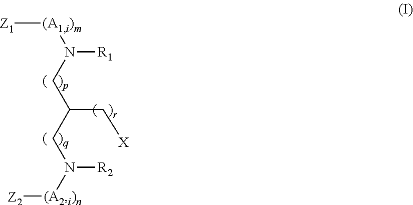
wherein i=1,2, . . . , m,
wherein j=1,2, . . . , n,
wherein n and m are independently an integer in the range of 0-3, with the proviso that the sum n+m is in the range of 1-4,
wherein p, q, and r are independently an integer in the range of 0-6,
A11, . . . , A1m, and A21, . . . , A2n are independently selected from α-amino acid moieties, β-amino acid moieties, α-amino sulphonic acid moieties, and β-amino sulphonic acid moieties,
Z1 and Z2 are independently selected from hydrogen, C₁₋₆ alkyl, carboxylic acid moieties (Z—C(═O)—) and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl, R1 and R2 are independently selected from hydrogen and C₁₋₆-alkyl; and
X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl and Hal is a halogen; and the total molecular weight of said ligand (excluding “X” and any linker) being 200-2000 g/mol.

2. The process according to claim 1, wherein
A11, . . . , A1m and A21, . . . , A2n independently are selected from glycine, L-proline, L-arginine, L-tyrosine, D-glutamine, D-tyrosine, L-valine, L-cysteine, L-histidine, and L-leucine;
Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl;
R1 and R2 are independently selected from hydrogen and C₁₋₆-alkyl; and
m is 0 or 1, n is 0 or 1, (p,q) is (0,1), (0,2), (0,3), (0,4), (1,0), (2,0), (3,0), or (4,0), and r is 0.

3. The process according to claim 1, wherein the ligand has the general formula (II),

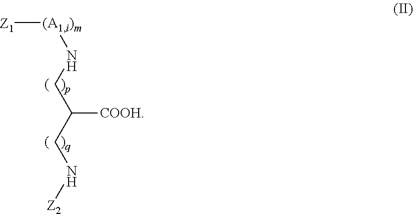

4. The process according to claim 3, wherein
p and q are independently an integer in the range of 0-6,
wherein i=1,2, . . . , m, and m is an integer in the range of 1-4, such as 1-3, e.g. 1-2; A11, . . . , A1m independently are selected from glycine, L-proline, L-arginine, L-tyrosine, D-glutamine, D-tyrosine, and L-valine; and
Z1 and Z2 are independently selected from 3,3-diphenyl-propionyl, 3,5-di-tert-butyl-4-hydroxy-benzoyl, mono-methyl-phthalyl, 3-(3,5-di-tert-butyl-4-hydroxyphenyl)propionyl, benzoyl, 2-thiophene-acetyl, salicylyl, 4-tert-butyl-benzoyl, 3,5-dimethoxy-2-naphthyl, naphthyl, phenyl-acetyl, and thianaphthenyl.

5. The process according to claim 4 wherein the ligand is selected from (1)-(12):

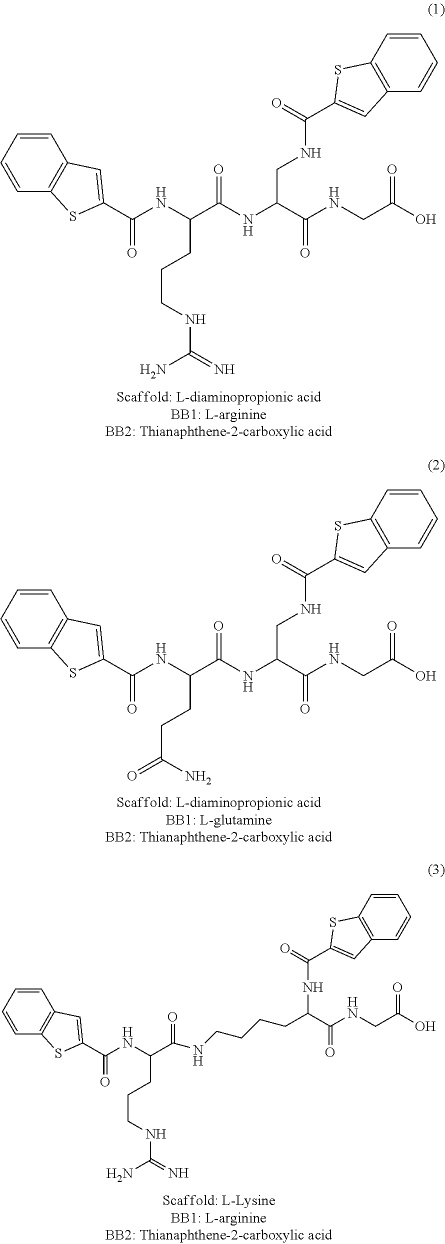

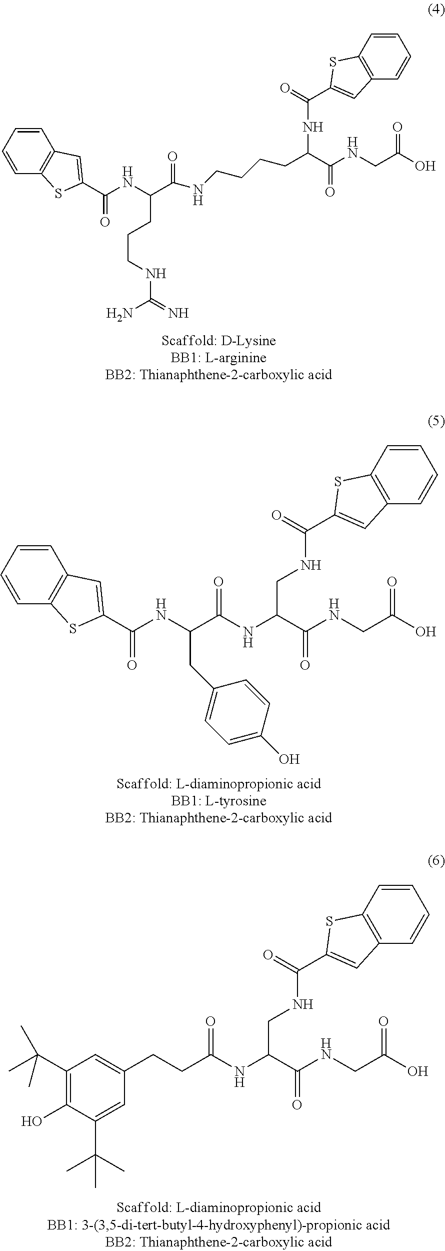

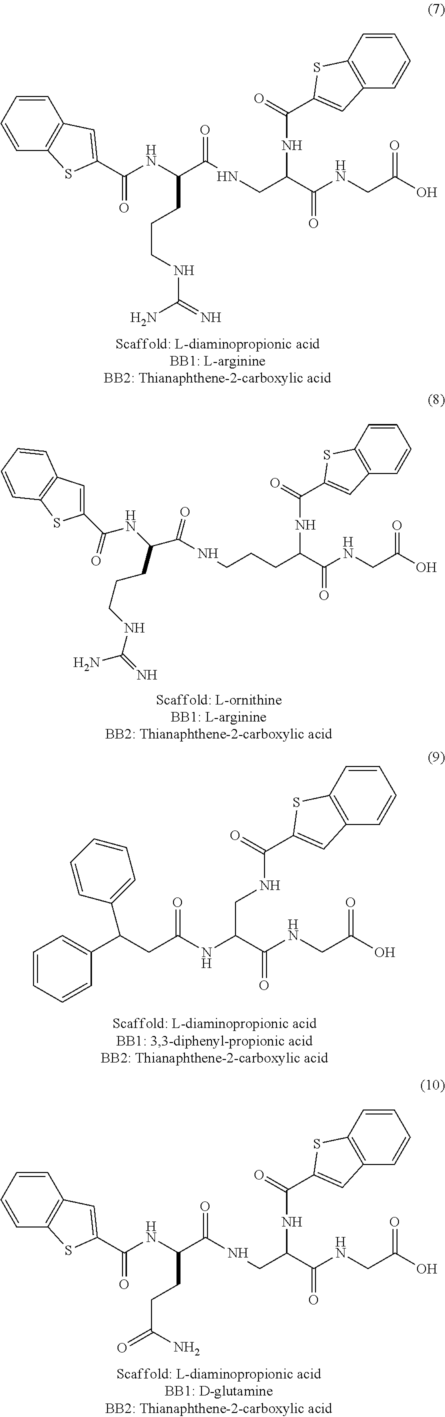

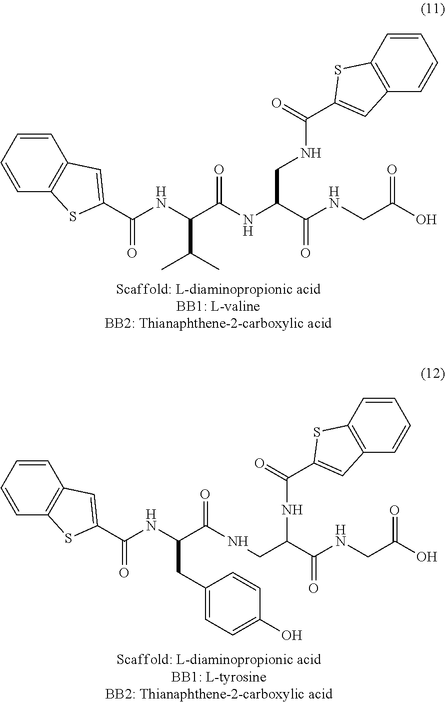

6. The process according to claim 1 wherein the ligand has the general formula (III)

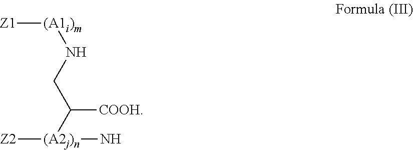

7. An affinity resin comprising a solid phase material having covalently immobilized thereto one or more ligands of the formula 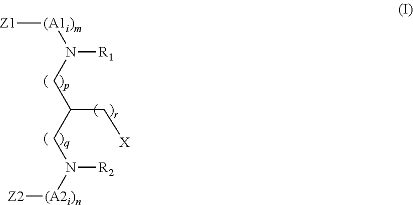
wherein i=1,2, . . . , m,
wherein j=1,2, . . . , n,
wherein n and m are independently an integer in the range of 0-3, with the proviso that the sum n+m is in the range of 1-4,
wherein p, q, and r are independently an integer in the range of 0-6,
A11, . . . , A1m, and A21, . . . , A2n, are independently selected from α-amino acid moieties, β-amino acid moieties, α-amino sulphonic acid moieties, and β-amino sulphonic acid moieties,
Z1 and Z2 are independently selected from hydrogen, C₁₋₆ alkyl, carboxylic acid moieties (Z—C(═O)—), and sulphonic acid moieties (Z—S(═O)₂—), wherein Z is selected from hydrogen, optionally substituted C₁₋₁₂-alkyl, optionally substituted C₃₋₁₂-cycloalkyl, optionally substituted C₁₋₁₂-alkenyl, optionally substituted C₁₋₁₂-alkynyl, optionally substituted aryl, optionally substituted heteroaryl and optionally substituted heterocyclyl,
R1 and R2 are independently selected from hydrogen and C₁₋₆-alkyl; and
X is the group for attachment of the ligand to the solid phase material, either directly or via a linker, X being selected from carboxylic acid (—COOH), a carboxylic acid ester (—COOR), a carboxylic acid anhydride (—COOCOR), a carboxylic acid halide (—COHal), sulphonic acid (—S(═O)₂OH), a sulphonyl chloride (—S(═O)₂Cl), thiol (—SH), a disulphide (—S—S—R), hydroxy (—OH), aldehyde (C(═O)H), epoxide (—CH(O)CH₂), cyanide (—CN), halogen (-Hal), primary amine (—NH₂), secondary amine (—NHR), hydrazide (—NH═NH₂), and azide (—N₃), wherein R is selected from optionally substituted C₁₋₁₂-alkyl and Hal is a halogen; and the total molecular weight of said ligand (excluding “X” and any linker) being 200-2000 g/mol.

8. The affinity resin according to claim 7, wherein the ligand has the general formula (II),

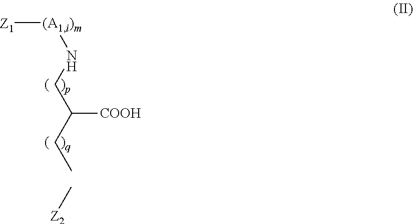

9. The affinity resin according to claim 7, wherein the ligand has the general formula (III)

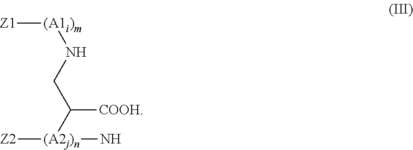

10. An affinity ligand selected from the group consisting of ligands (1)-(12) illustrated herein.
